(19) United States
(12) Patent Application Publication
(10) Pub. No.: US 2007/0248573 A1
(43) Pub. Date: Oct. 25, 2007

(54) Chimeric Bacteriophages, Chimeric Phage-Like Particles, and Chimeric Phage Ghost Particles, Methods for Their Production and Use

(76) Inventor: Joseph Sturino, Milwaukee, WI (US)

Correspondence Address: NIXON & VANDERHYE, PC, 901 NORTH GLEBE ROAD, 11TH FLOOR, ARLINGTON, VA, 22203, US

(21) Appl. No.: 11/663,209

(22) PCT Filed: Sep. 19, 2005
(86) PCT No.: PCT/DK05/00587

§ 371 Date: Mar. 19, 2007

Related U.S. Application Data
(60) Provisional application No. 60/610,530, filed on Sep. 17, 2004.

(51) Int. Cl. A61K 45/00; A61K 39/12; A61P 31/00; C07K 14/00; C12P 21/02

(52) U.S. Cl. 424/93.6; 424/204.1; 424/278.1; 435/69.1; 435/69.7; 530/350

(57) ABSTRACT

The objective of the present invention is to provide chimeric phage-derived particles, that may be used as safe food grade vehicles to for presenting various factors (e.g. antigens, virulence proteins, receptors, ligands, etc.) for living cells. In addition to at least one normal phage or virus component the particles comprise at least one additional factor that is not encoded by the genetic material of the chimeric particle. Applications for such particles include, but are not limited to, vaccine development, pathogen neutralization, chemical binding and/or neutralization (e.g. toxins), and competitive exclusion. In addition, this technology may be used to extend the retention time of phage particles during phage therapy and/or specifically target a given particle for a biofilm.

### BACKGROUND OF THE INVENTION

Previously, phages displaying epitopes have been administered subcutaneously (Nature Biotechnology 18 (2000), 873-6). This article discloses that the filamentous bacteriophage fd can display multiple copies of foreign peptides in the N-terminal region of its major coat protein, and that such a construct is usable for subcutaneous immunization. Nucleic Acids Research 25 (1997), 915-916 also discloses hybrid virions of bacteriophage fd that display two different peptides. It is suggested that such a construct has many potential advantages in exploring vaccine design.

A recent trend in vaccinology has been the development of mucosal vaccines for the treatment and prevention of diseases, including (i) cancer, (ii) hypersensitivity to allergens (including the suppression of atopic disease) and (iii) infection by pathogenic microorganisms (including viruses, bacteria, fungi, and protists) in humans and animals.

The principle sites of antigen exposure include the conjunctiva, the gastrointestinal tract, the respiratory tract, and the urogenital tract. Thus, the mucosal epithelia that line these sites constitute active barriers between the internal and the external environment and serve as an organism's primary defense against infection (Salminen et al., 1998, 2001). Since pathogens generally contact and/or colonize mucosal epithelia before becoming systemic, one of the most obvious advantages of mucosally administered vaccines over traditional injectable vaccines is that their route of introduction more closely mimics the means by which these anti-gens normally enter the host. As a result, it is believed that mucosal vaccines will trigger robust responses from both the innate and adaptive arms of the immune system and thus may afford more complete protection relative to vaccines administered by other means, especially injection. One of the problems with traditional injectable vaccines is that they mimic an ongoing systemic infection and, as a result, generally bypass large portions of the immune system (especially the innate immune system) that are normally involved in the body's first exposure to non-self antigens.

There are several studies showing that mucosal administration of vaccines offers better protection against pathogens that enter the body via mucosal tissues than that offered by conventional vaccines (Davis, 2001).

To produce live attenuated vaccines, derivatives of known pathogens have been engineered to exhibit reduced virulence in their host. Such strains are routinely used as the active component of live attenuated vaccines. Live attenuated vaccines generally confer excellent immunity to the host, and may elicit both humoral and cellular immunities that may last a lifetime. These vaccines are generally protective following the administration of a few or even a single dose(s). However, live attenuated vaccine formulations carry significant risks and a wide variety of drawbacks are associated with their usage. Among the most serious of these is the possibility that the attenuated strain may revert to virulence in vivo and cause the disease that the vaccine was intended to prevent. Further, infection by vaccine strains may be transmitted to immunocompromised or unvaccinated individuals, which includes children and the elderly. Furthermore, adverse reactions and even serious adverse reactions, which include death, are sometimes associated with live attenuated and inactivated vaccines, in part because the vaccine preparations contain a multitude of poorly defined components normally associated with the given microorganism. For these and other reasons, attenuated vaccines are generally inappropriate for use in children, the elderly, and immunocompromised individuals, including those infected with HIV. Especially in these populations, but also in healthy adult populations, physicians increasing choose to prescribe safer alternatives (e.g. subunit vaccines); even if such alternatives may exhibit somewhat reduced efficacy (Foss and Murtaugh, 2000). The FDA acknowledges these risks, and continues to adopt more stringent guidelines regarding the development of new vaccine products. New FDA guidelines require companies to more accurately define the composition of their vaccine products, which makes the development of new live attenuated and killed vaccines much more difficult. These trends have prompted the biotechnology and pharmaceutical sectors to largely forgo the development of new varieties of live attenuated vaccines and instead develop new vaccine formutations (e.g. subunit vaccines) that may have a better-defined composition and safety profile.

The genes encoding antigenic factors are associated with the live and inactivated microorganism components of vaccines, regardless of their method of administration. There is a very real and significant risk of horizontal transmission of these genes, which include but are not limited to genes encoding virulence and antibiotic resistance factors, to the host's resident microflora by transduction, transformation, and/or conjugation. The frequency by which this transfer may occur likely depends not only on the composition of the vaccine, but also the means by which it is administered. Since the bloodstream is essentially devoid of native microflora in healthy individuals, intravenous (IV) administration of even high levels of live microorganisms likely represents a very small risk of horizontal transmission. As a result, this means of administration represents a safer (albeit more invasive) route to deliver protective antigens (although adverse reactions associated with injection site infections, etc. are still a concern). In contrast, the risk of transfer is likely significant when live vaccines are administered to niches containing high levels of native resident microflora of heterogeneous composition (e.g. the gastrointestinal tract, the vagina, the respiratory tract etc.). As a result, exposure of the mucosal membranes (e.g. intragastric, intravaginal, intranasal, etc.) of live attenuated, live recombinant, and inactivated vaccine microorganisms represents a risk for the transmission of virulence and antibiotic resistance genes, which may result in the emergence of new pathogens.

With regards to the horizontal transfer of genes coding for antibiotic resistance and/or virulence factors, the administration of plasmid-encoding vaccine strains is the most disconcerting, as these mobile DNA elements have a long history of promiscuous transmissibility, even between genera. Plasmids are routinely transmitted by transduction, transformation, and conjugation. Evidence has begun to accumulate to support the supposition that DNA encoding virulence factors, including antibiotic resistance markers, can be transferred from foreign microorganisms to the native microflora of both humans and animals. Wilcks et al., (2004) demonstrated that plasmids from genetically modified plants persist in the gastrointestinal (GI) tract of rats. Critically, these plasmids were found to be intact and able to transform electrocompetent Escherichia coli, indicating that they were available for uptake by intestinal bacteria. Similarly, Blake et al. (2003) demonstrated that antibiotic resistance genes, a type of virulence factor, are exchanged between E. coli O157, Salmonella species (spp.), and strains commensurate with Escherichia coli. Further, Mercer et al. (1999) showed that DNA released from bacteria or food sources within the mouth has the potential to transform naturally competent oral bacteria. Although these studies were not performed with vaccines specifically in mind, these studies clearly illustrated that these types of transmissions do occur, and suggest that alternative methods for the preparation of vaccines should be developed in order to further limit the introduction of virulence factors to the native microflora of the host.

There is a general concern over the use of genetically modified organisms (GMOs) in foods and agriculture. Once introduced into a system, it is feared that the recombinant genetic material associated with GMOs will be disseminated into the environment and thus confer novel (and perhaps dangerous) characteristics to endogenous species. At this time, the dangers associated with GMOs appear greatly exaggerated, however it can no longer be disputed that large-scale introduction of genetic material may result in the transmission of these genes into the native species.

Accordingly there is a call for safe technologies that reduce these risks while maintaining their benefits of vaccines.

### SUMMARY OF THE INVENTION

It is an object of the present invention to provide a composition comprising chimeric phage-derived particles (such as chimeric bacteriophage (i.e. phage) particles, chimeric phage-like particles or chimeric phage ghost particles) that, in addition to the phage-encoded factors, displays one or more additional factors (e.g. antigens, allergens, virulence proteins, receptors, ligands etc., especially a heterologous antigen). In particular, a composition of chimeric particles that is safe for the inoculation of humans and/or animals is considered.

One way of making the particles safe is by producing the particles in a safe host cell. Thus the invention, in a first aspect, pertains to a method of production of a chimeric phage-derived particle (and to a method of production of a composition comprising said particle), said method comprises introducing into (eg. by transfection, infection and/or otherwise transformation) a safe host cell one or more (such as 2, 3, or 4) genetic elements, which alone or in combination encodes the phage-derived particle. The safe host cell may be selected from the group of bacteria consisting of:
bacteria the use of which have been evaluated by the United States Food and Drug Administration, Center for Veterinary Medicine to be generally recognized as safe (GRAS) and especially bacteria which appeared on the Partial List Of Microorganisms And Microbial-Derived Ingredients That Are Used In Foods published on Sep. 16, 2005 at http://www.cfsan.fda.gov/˜-dms/opa-micr.html; and
bacteria that, according to the list published by the European Food and Fed Cultures Association and International Dairy Federation (EFFCA/IDF) (Mogensen et al. (2002)) are microorganisms with a documented history of use in food without adverse effects; and
bacteria that were accepted by the Danish Veterinary and Food Administration as food cultures for use in food in Denmark on Sep. 16, 2005.

It should be understood that when only one genetic element is introduced into the host cell, said genetic element should encode all components of the chimeric phage-derived particle. It is presently preferred that the element is constructed in such a way that it is not incorporated in the chimeric particle, or it is removed from (or inactivated in) the particle by eg. chemical treatment. When two or more elements are introduced into the host cell, it is presently preferred that one element (eg. being a phage) encodes a wild-type phage, while an other element (eg. being a plasmid) encodes a hybrid of a wild-type phage protein and the polypeptide to be displayed on the phage surface.

Also according to the invention, the problem of providing a composition of chimeric phage-derived particles (such as chimeric phage particles, chimeric phage-like particles or chimeric phage ghost particles) that is safe for human and/or animal intake may be solved by using bacteria and their associated phages with a proven track record of being generally recognized as safe (GRAS) to produce chimeric particles that do not encode the gene for said additional surface displayed factors.

Thus in a further aspect, the invention pertains to a method of production of a composition comprising chimeric phage-derived particles that comprise at least two non-identical surface displayed proteins, said method comprising the steps of: (i) obtaining at least two genetic elements wherein at least one of said genetic elements (the founder genetic element) comprises a substantial part of a phage genome and wherein the at least one other of said genetic elements (the trans-complementing genetic element) codes for the synthesis of at least component (the additional component) that is directed to the surface of said particle during the assembly and/or release of the particle; (ii) transfecting, infecting and/or otherwise transforming a suitable bacterial host cell with said two or more genetic elements; (iii) culturing said bacterial host cell under conditions that permit the expression of phage structural proteins encoded by said founder genetic element and the expression of said at least one additional component that is directed to the surface of said particle during the assembly and/or release of the particle; (iv) subjecting said culture of bacterial host cells to conditions that results in formation of chimeric phage-derived particles that comprise at least one additional component that is encoded by the trans-complementing genetic element but do not comprise the sequence encoding for, at least part of, said at least one additional component that is encoded by the trans-complementing genetic element; and (v) obtaining a composition comprising chimeric phage-derived particles.

As will be described in further detail below such chimeric particles may be isolated from the described composition. Accordingly, a second aspect the invention relates to a chimeric phage-derived particle that in addition to at least one normal phage component display or comprise at least one additional component, said at least one normal phage component being coded by a genetic element that comprises a substantial part of a phage genome and said at least one additional component being encoded by a different genetic element, the particle is further characterized in that it does not comprise the sequence encoding for at particle is further characterized in that it does not comprise the sequence encoding for at least part of said at least one additional component.

Such particles will possess a safety profile, which may warrant certain regulatory advantages that could facilitate rapid approval by the appropriate regulatory agencies. Consequently, further aspects of the invention relate to the use of a chimeric phage-derived particle according to the invention to produce a vaccine, a composition for phage therapy, for the treatment of allergies, for the use as a biocontrol agent and other uses that will be further described.

Possible applications include but are not limited to the generation of chimeric particles that surface display one or more factors that:

. . . allow the particles to competitively exclude pathogens or other non-beneficial microflora (e.g. those that reduce feed efficiency) from mucosal surfaces (e.g. the gastrointestinal tract).

. . . act as a generic adapter (e.g. polyhistidine tag) for the specific and non-covalent attachment to a second adaptor component (e.g. mouse anti-polyhistidine antibody) that is covalently conjugated to a heterologous bioactive molecule or complex (e.g. alkaline phosphatase).

. . . stimulate the immune system and enable the particles to act as a vaccine and/or immunostimulatory adjuvant. Since certain native phage proteins may be highly antigenic, the phage-based delivery vehicle is also contemplated to act as an adjuvant to stimulate the immune system. Further, the size of the particles will also enhance the immune response by stimulating the action of antigen presenting cells (APCs). Multivalent vaccine preparations may be formulated either by (i) surface-displaying two or more antigens on a single particle or (ii) using two or more distinct particle species in combination. Alternative factors could be used to specifically enable the targeting of T- or B-lymphocytes to deliver their antigenic cargo (i.e. enable the particles to act as a smart vaccine).

. . . extend the retention time of phage particles on mucosal epithelia and/or their cognate biofilms in vivo. Furthermore the invention could be used to provide industrial surfaces or biofilms (i.e. for use in Phage Biocontrol).

. . . selectively enable the particles to deliver cytotoxic payloads to kill cancerous cells, pathogens, or infected host cells. For example, one surface-displayed factor could confer selective specificity for a specific type of cancerous cell (or a pathogen), while a second factor could act as a cytotoxin. It may be possible for the cytotoxin to be sequestered from the host (e.g. in a hydrophobic pocket) until after it has bound an appropriate target. Alternatively, these particles could be used to specifically target lymphocytes to deliver their cytotoxic cargo (e.g. to selectively kill HIV-infected lymphocytes).

. . . allow the particles to specifically bind and coat a pathogen in vivo (e.g. in the gastrointestinal tract), thereby neutralizing it and allowing it to be passaged without causing disease.

. . . facilitate the purification of the chimeric particles from the non-chimeric particles, cellular debris, and spent culture medium (e.g. polyhistidine tag).

### DEFINITIONS

As used herein the term “lactic acid bacterium” (LAB) designates a gram-positive, catalase negative facultative anaerobic or microaerophilic bacterium that ferments sugars with the production of acids including lactic acid as the predominant fermentation product. The industrially most useful lactic acid bacteria are found among Lactococcus spp., Streptococcus spp., Lactobacillus spp., Leuconostoc spp., Pediococcus spp., Oenococcus spp., Brevibacterium spp., Enterococcus spp. and Propionibacterium spp. Additionally, lactic acid producing bacteria belonging to the group of the strict anaerobic bacteria, bifidobacteria, i.e. Bifidobacterium spp. that are frequently used as food starter cultures alone or in combination with lactic acid bacteria, are generally included in the group of lactic acid bacteria.

A “phage” or “bacteriophage”, as used herein, relates to the well-known category of viruses that infect bacteria. Phages includes DNA or RNA sequences encapsidated in a protein envelope or coat (“capsid”). Phage may be transmitted to host cells via a variety of processes, including infection, transformation, transfection and/or conjugation.

A “virus”, as used herein, means the well-understood term of the art, as well as other species that may be derived from phage or viruses as are understood and known by those of skill in the art.

A “host cell”, “host” or “host organism”, as used herein a is used interchangeably to describe a suitable bacterial cell in which the founder genetic element and the trans-complementing genetic element are introduced—and in which the phage from which the founder genetic element originates can replicate.

A “culture”, as used herein, relates to populations of bacterial cells that results from the bacterial growth in any medium and includes fermented feed and food products such as fermented dairy products, meat, fish, fruit and/or vegetable products.

A “phage base plate”, as used herein, relates to a structure responsible for linking phage tail fibers and/or spikes to the phage.

A “phage collar”, as used herein, relates to a structure responsible for linking head and whiskers to the tail structure.

A “phage tail fiber”, as used herein, relates to a filamentous structure found at base of a phage particle. The phage tail fibers are typically responsible for recognition of suitable host cell(s).

A “phage prohead”, as used herein, relates to the phage head structure prior to phage genome encapsidation and joining to tail/and or collar components.

A “phage head”, as used herein, relates to an icosahedral-structure that houses the phage genome.

A “phage tail”, as used herein, relates to a tubular structure that channels phage genome as it exits the phage head and enters the host cytoplasm.

A “phage whisker”, as used herein, relates to filamentous structures that may or may not be used to sense the phage's physiochemical environment.

A “probiotic composition”, as used herein, relates to a composition that comprise probiotic organisms defined as live microorganisms that when administered in adequate amounts confer a health benefit on the host (FAO/WHO report, October 2001 http://www.mesanders.com/probio_report.pdf.). Conventionally, and also in this context, this definition also included the following statement: “ . . . that the benefit goes beyond simple nutrition’. (i.e. they are doing more than being digested for the number of calories that they contain). In the present context phage particles, but not phage-like or phage ghost particles, is considered as live microorganisms as they may infect host cells.

In the present context, the term “substantial part of a phage genome” is to be understood a part of the a phage genome comprising at least 70% of the genetic loci necessary to form a functional phage genome, preferably at least 80%, more preferably at least 90%, or even more preferably all of the genes and other gene sequences that are required to form a functional phage genome.

A “functional phage genome”, as used herein, relates to a DNA or RNA molecule or set of DNA or RNA molecules that when introduced into at suitable bacterial cell, in certain situations together with one or more phage coded factors, are able to direct a complete infectious cycle that results in formation of phage particles. In a preferred form, this or these DNA or RNA molecules will contain an origin of DNA replication derived from the parent phage.

By the term “component” is understood a part of which a phage (or phage like or phage ghost particle) is made. The term includes a protein encoded by the phage genome.

An “additional component”, as used herein the term refers to a component encoded by the trans-complementing genetic element that is directed to the surface of the chimeric phage-derived particles (such as chimeric phage particles, chimeric phage-like particles or chimeric phage ghost particles) during the assembly and/or release of the particles.

“Isolate”, as used herein with respect to chimeric phage-derived particles refers to a procedure wherein the particles are removed from their original environment (e.g. such as the particle-producing culture of bacterial host cells).

The term “antibody” is used herein in the broadest sense and specifically covers, intact monoclonal antibodies, polyclonal antibodies, multispecific antibodies (e.g. bispecific antibodies) formed from at least two intact antibodies, and antibody fragments as long as they exhibit the desired biological activity.

As used herein an “antigen or antigenic determinant” relates to the portion of an antigen molecule that determines the specificity of the antigen-antibody reaction.

An “episome”, as used herein, relates to a type of plasmids that can reversibly integrate into the cell's chromosome.

“Animal”, as used herein, relates to vertebrates such as humans and animals including fishes, birds such as e.g. chickens, turkeys and ostriches, and mammals such as e.g. dogs, cats, rabbits, cattle, pigs, buffaloes, camels, deer, antelopes, giraffes, sheep, goats, horses, donkeys, elephants, monkeys and chimpanzees.

“Holin”, as used herein, relates to a protein that is typically expressed from the phage genome in the late stages of phage infection. Holin proteins form a pore in the cell membrane and allow lysin or lysozyme proteins to gain access to the cell wall peptidoglycan, which results in cell lysis a release of progeny phage particles.

“Capsid”, as used herein, is defined as the external coat of a virus particle. Also the coat of “phage ghost” and “chimeric phage-like particles” is referred to as the capsid.

“Lysin”, as used herein, relates to a murine hydrolase capable of degrading the bacterial cell wall to allow phage release (for a review see Young et al., 2000).

“Neutralization of biological toxins”, as used herein, relates to the act of reducing or eliminating the toxicity of specific toxins through the interaction of one or more chimeric phage particle(s) with the agent(s) of interest. Without being limited to a particular explanation or theory, the neutralization of biological toxins can be explained by the sequestering of the biological agent from its intended host receptor; thereby eliminating the cascade of events that result in adverse reaction to said agent.

“Neutralization of a pathogen”, as used herein, relates to the act of reducing or eliminating the toxicity of specific biological agents through the interaction of one or more chimeric phage particle(s) with the agent(s) of interest. Without being limited to a particular explanation or theory, the neutralization of the pathogen can be explained by the sequestering of the biological agent from its intended host receptor or niche; thereby eliminating the cascade of events that result in adverse reaction to said agent. The neutralization may also prevent nutrient uptake “Non-desirable microorganisms”, as used herein, relates to a microorganism or group of microorganisms that, when present in specific niches within a human or animal, reduces the overall fitness and or biochemical efficacy of the colonized human or animal, without causing a specific disease state. For example, certain species of bacteria cause excessive gas (flatulence) in ruminant animals. These cows are not sick, but they exhibit reduced efficient feed assimilation.

“Phage structural proteins”, as used herein, relates to proteins and/or peptides that comprise the component(s) of the phage's capsid and/or components thereof.

“Phage infection”, as used herein, relates to the state whereby the phage's genome has been successfully introduced into the cytoplasm of host bacterium. Infection, as used herein, is independent of “phage replication”.

“Phage replication”, as used herein, relates to the synthesis, processing, and assembly of phage components into mature phage particles. Phage replication requires gene expression in the host and is independent of the infectiousness of the mature particle.

“Phage therapy”, as used herein, relates to the utilization of phages to eliminate and/or reduce the number of pathogenic and/or otherwise non-desirable microorganisms present in humans and/or animals though any number of mechanisms that comprise but are not limited to predation, competitive exclusion, pathogen neutralization, or any combination thereof. Phage therapy may also be used to enrich for desirable microorganisms through the reduction or elimination of non-desirable microorganisms, including pathogens, and phage therapy may be used as an alternative treatment for the acute and/or chronic disease states.

“Founder particle”, as used herein, relates to the naturally occurring phage isolate from where the founder genetic element was derived.

“Founder genetic element”, as used herein, relates to a genetic element that comprises a substantial part of a phage genome of the founder particle.

“Virus or phage biocontrol”, as used herein, relates to the utilization of viruses or phages to eliminate and/or reduce the number of pathogenic and/or otherwise non-desirable microorganisms present in a defined environment through any number of mechanisms that comprise but are not limited to predation, competitive exclusion pathogen neutralization, or combination thereof. Virus or phage biocontrol may also be used to enrich for desirable microorganisms through the reduction or elimination of non-desirable microorganisms, including pathogens. As used herein virus or phage biocontrol does not apply to living human or animal systems, it refers solely to non-therapeutic applications (e.g. use in packaging, to spray on animal carcass, etc.).

“Plasmids”, as used herein, are autonomously replicating molecules, which are often composed of double stranded DNA, that exist in cells as extra chromosomal elements. Generally, plasmids contain a limited number of genes and often encode one or more proteins required for their own replication. In most situations, plasmids are non-essential and encode dispensable functions that augment certain cellular metabolic capacities.

“Prophage”, as used herein, relates to a relatively passive form of a bacteriophage replication whereby the phage genome is incorporated into the bacterial chromosome without causing death of the host cell.

“Suitable host cell”, as used herein, relates to a cell that can be transfected, infected and/or otherwise transformed with the founder genetic element and the trans-complementing genetic element and support the expression of said genetic elements and assembly of chimeric phage particles, chimeric phage-like particles, or chimeric phage ghost particles.

“Trans-complementing genetic element” is a genetic element that codes for at least one component that is not coded by the founder genetic element, and preferably does not comprise a substantial part of a phage genome. Typically the “trans-complementing genetic element” is a plasmid. Importantly, a helper-phage defined herein as a normal wild-type version of the phage, which grows along with a specialized phage (i.e. the founder genetic element) and supplies whatever functions are necessary for generating phage particles is not considered as a “trans-complementing genetic element”.

“Virus-like particles” (VLPs) are defined as self-assembling, non-replicating, nonpathogenic, and genome-less particles that are comparable in size to intact virions, but are composed by far less components than virions are. In practice, VLPs are generally comprised of a single phage or virus capsid protein (capsomer), which is fused to an antigen/epitope of interest. This fusion protein is generally expressed from a plasmid in the absence of the phage genome from which the gene was isolated. VLPs are thus an engineered and plasmid-driven technology. Although VLPs and phage ghosts share some important characteristics (e.g. neither contain genetic material), they are functionally and biologically distinct by definition and in their manner of preparation.

“Phage-like particles” are defined as particles derived from a phage particle that are comparable in size to intact virions, are composed by a number (typically fewer) of components similar, but not necessarily identical, to the intact virion from which they are derived and may even contain part of the founder genetic element.

“Phage ghosts” are defined as bacteriophage-derived empty protein coats. Phage ghosts may be produced, through the induction of a defective prophage by interfering with a lytic infection due to the expression of one or more factors, or in some cases through the chemical treatment (e.g. osmotic shock) of intact (i.e. genome-containing) phage particles. In the present context, the term “gene” is used to indicate a DNA or a RNA sequence that is involved in producing a polypeptide chain and that includes regions preceding and following the coding region (5′-up-stream and 3′-downstream sequences). The 5′-upstream region comprises a regulatory sequence, which controls the expression of the gene, typically a promoter. The 3′-downstream region comprises sequences that are involved in termination of transcription of the gene.

In the present context, the term “helper phage” is used to describe a normal wild-type version of the phage, which grows along with a specialized phage and supplies whatever functions are necessary for generating phage particles.

“Unrelated peptide sequence,” describes a peptide sequence that is different from the peptide sequence that directs the fusion protein to the surface of the phage.

In the present context, the term “chimeric particles” is used to describe chimeric phage particles, chimeric phage-like particles or chimeric phage ghost particles consisting of proteins and/or other components that are encoded by at least two different elements, in casu, components that are encoded by the founder genetic element(s) and by the trans-complementing genetic element(s).

By the term “chimeric phage-derived particle” is understood a particle derived from a phage in the sense that it displays at least one additional (heterologous) component at its surface, (preferably in addition to at least one normal phage-encoded component). The particle may be selected from the group consisting of a chimeric phage particle, a chimeric phage-like particle and a chimeric phage ghost particle, or from a particle that is obtainable by a method of the invention.

“Safe microorganisms” are defined as microorganisms that are GRAS status for use in food or feed and/or are considered microorganisms with a documented history of use in food without adverse effect by appropriate regulatory agencies. The term “safe microorganism” is used interchangeable with the term “safe host cell”.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DETAILED DISCLOSURE OF THE INVENTION

To the best of the knowledge of the inventor, all attempts to exploit chimeric virus or phage as vehicles for displaying one or more components have been based on virus/host cell systems that are potentially pathogenic. In addition, the prior art on chimeric phages fails to segregate the one or more additional factors that are found on the surface of the chimeric phage, especially if one of these factors is a fusion protein, from the gene that encodes those one or more factor(s).

In order to provide the safest possible composition of chimeric phage-derived particles the present invention describe a method to produce such chimeric particles that to a large extent is based on virus/host cell systems that are generally recognized as safe. Examples of such systems are host organisms that possess GRAS status for use in food or feed and/or are considered microorganisms with a documented history of use in food without adverse effects by appropriate regulatory agencies (herein referred to as “safe microorganisms”). Although the phages are naturally associated with such host cells, they are not explicitly considered either “GRAS” or ascribed a “safe microorganism” status by the relevant regulatory agencies. A significant number of reports prove that many if not all of the “safe microorganisms” are subjected to phage infection at a regular basis. Such phages are naturally and inevitably associated with their specific host cells and the food products that are made using these bacteria. Thus, a status as “safe microorganism” must be based on a documented history of use in food without adverse effects that necessarily comprise both the host cell and their associated phages.

As an important extra safety precaution, a presently preferred embodiment of the method of the present invention further devises that the at least one gene that codes for the at least one additional component that is encoded by the trans-complementing genetic element and that is displayed on the surface of the chimeric phage-derived particles is not contained in the particles. This separation of the gene from the chimeric phage can conveniently be accomplished by ensuring that the genome of said host cell and/or said founder particle and/or said trans-complementing genetic element possess intrinsic quality to ensure that the genetic material from the trans-complementing genetic element do not associate with said chimeric particles. One example of such an intrinsic quality is expressed by most bacterial plasmids. If the trans-complementing genetic element is a plasmid, the gene(s) residing on the plasmid will not be transferred to the chimeric particles unless special packaging signals are present on the plasmid. Similarly, if the trans-complementing genetic element is integrated into the bacterial genome e.g. via a recombinant transposon, it is highly unlikely that the gene(s) residing on the trans-complementing genetic element will be transferred to the chimeric phage particles. However, it is possible to envision even further measures that reduce the likelihood of said gene(s) being transferred to the chimeric particles. It is contemplated that the genome of the host cell and/or said founder particle and/or said trans-complementing genetic element could be engineered to express some factor (e.g. antisense RNA or transdominant protein expression, conditional mutation, etc.) that ensure the segregation of the gene from said chimeric particles. Thus, even though the chimeric particles may display part of virulence factors or toxins the risk of horizontal transference of “adverse” genetic elements coding for an adverse component is virtually nil.

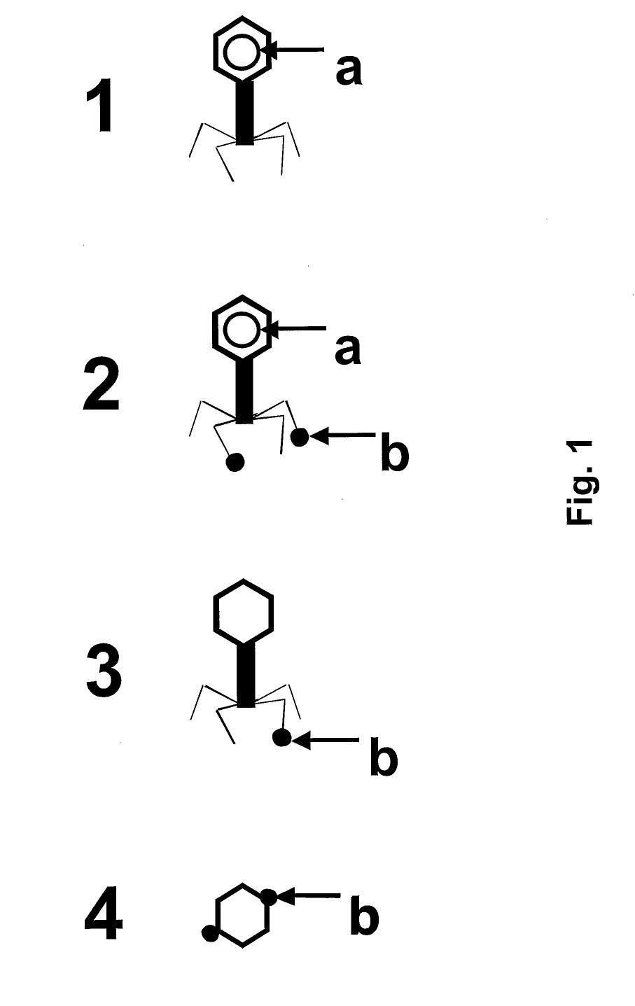

FIG. 1 illustrates the principal types of particles that results from this method.

It should to be noted that particles produced by the method disclosed herein will result in chimeric particles that in addition to the components encoded by the founder genetic element that comprises a substantial part of a phage genome also comprise one or more additional factors encoded by the trans-complementing genetic element. One or more of these additional factors may be displayed on a single particle, as can multiple units of the same factor(s). Surface displayed factors may be incorporated into the tail fibers (as shown), head, tail, or any other structure associated with the phage particle. In most applications, the particles need not be intact to be efficacious as vehicles for the displayed factors. The particles do not contain genetic material of the trans-complementing genetic element that codes for the additional factor(s). Thus, although the particles are chimeric, they are not genetically recombinant. As a result, legislative restrictions pertaining to recombinant GMO technology appears not apply to these products.

It is an important aspect of the present invention that the founder genetic element comprises a substantial part of a phage genome. This is in contrast to e.g. WO04003143 wherein the virus-like particles contains no naturally occurring phage capsid components but is composed of a viral coat protein translationally fused to exogenous peptide sequences.

The founder genetic element and the trans-complementing genetic element may be introduced into the host cells simultaneously in one step, however, for technical reasons it is often preferred that the two genetic elements are introduced into the host cells by a sequential method comprising the steps of: (i) transfecting, infecting and/or otherwise transforming a suitable host cell with said at least one trans-complementing genetic element; and (ii) transfecting, infecting and/or otherwise transforming the host cell with said founder genetic element. The advantages of this embodiment of the method are particularly striking when the founder genetic element is a complete phage genome coding for a naturally occurring infective phage. In the first step of this embodiment of the method, the trans-complementing genetic element, which is typically selected from the group of genetic element consisting of plasmids, transposons, prophages, prophage remnants, pesudophages, episomes, and phagemids is introduced into a host cells. Subsequently, a host cell that contains the transcomplementing genetic element is selected, characterized and expanded. A further advantage of this approach is that such transformed host cells can be kept for considerable time at low temperature and infected with different phages containing different founder genetic elements.

Although the founder genetic element is comprised of the genome of a phage isolate, especially one isolated from nature, it may code for only a few functional phage components. However in a preferred embodiment, the founder genetic element encodes several normal phage components resulting in chimeric phage-derived particles that comprise several normal phage components.

Whether the method described herein results in chimeric phage-derived particles depends on the genetic contents of the host cell genome, the founder genetic element and the trans-complementing element in addition to the precise experimental conditions.

The method will result in a chimeric phage if the founder genetic element is able to complete an infective cycle in the presence of at least one additional factor encoded a trans-complementing genetic element. Typically the founder genetic element will multiply during the process. A chimeric phage-like particle will result from introduction of a founder genetic element that does not code for all the factors encoded by the founder particle that are required to assemble a native founder particle and that are able to carry out a complete infective cycle. Finally, the production of chimeric phage ghost particles typically requires an additional processing event.

This additional processing event will normally be the result of one of two possible operations. The first option would be to use a mother phage that fails to incorporate its genome. This defect may be conditional or not. If it is conditional, then the phages will be able to encapsidate their genomes under one condition (e.g. temperature 1) but fail to encapsidate their genomes under a second condition (e.g. temperature 2). If they are not conditional mutants, then a factor must be supplied in trans in order to replicate the mutant phage. Regardless of the means, once replicated, these mutant phage particles can then be used to infect the host cells that already contain the trans-complementing genetic element described above. Once infected, the host cells will go on to synthesize tagged ghost particles devoid of DNA.

The second option would be to infect a host cells that already contain the trans-complementing genetic element that is expressing a second genetic construct that is designed to specifically prevent the encapsidation of the phage genome. This may be accomplished by any number of means, including (but not limited to) the expression of (i) natural phage resistance mechanisms (including, but not limited to abortive defense systems), (ii) the expression of antisense RNA specific for phage transcript(s) that encode one or more genome encapsidation factors; (iii) trans-dominant negative mutant derivatives of phage-encoded genome encapsidation factors (e.g. proteins); or (vi) factors that repress the expression of the genome encapsidation genes or inactivate their encoded proteins. Regardless of the means, once replicated, these mutant phage particles can then be used to infect the host cells that already contain the trans-complementing genetic element described above. Once infected, the host will go on to synthesize tagged ghost particles devoid of DNA.

Once the mature particles have been assembled, the host cells will be lysed either by the appropriate phage-encoded machinery, thereby releasing the intracellular contents (including particles) into the growth medium or, if the founder and the complementing genetic elements does not provide all necessary factors, by non-phage-induced lysis, including the physical disruption (e.g. sonication) and/or the addition of chemicals (e.g. phage lysins, lysozyme, etc.). In a preferred embodiment of the method, the particles are liberated from the host cells by a natural lytic process. This implies that the transformed host cells express the appropriate machinery required for assembly and release of the particles, i.e. that they express all necessary phage-encoded genes that direct particle assembly and release.

In this respect, the chimeric phage-like and the chimeric phage ghost particles of the present invention are different from VLPs in that the usual understanding may be defined as self-assembling, non-replicating, non-pathogenic, and genome-less particles that are comparable in size to intact virions, but are composed by far less components than virions are. In practice, VLPs are generally comprised of few, typically only a single phage or virus capsid protein (capsomer) that is fused to an antigen/epitope of interest. This fusion protein of VLPs is generally expressed from a plasmid in a host cell, which do not contain a substantial part of the phage genome from which the gene was isolated. VLPs are thus an engineered and plasmid-driven technology. Although VLPs and phage ghosts share some important characteristics (e.g. neither contain genetic material), they are functionally and biologically distinct by definition and in their manner of preparation. To the knowledge of the present inventor all, VLPs must generally be liberated from their cellular factories by human invention (e.g. by subjecting the host cells to sonication, French press, chemical lysis or similar procedures).

Once the phage particles have been released, the fermentate is treated to remove exogenous nucleic acids—including the recombinant fusion gene. Additional treatment steps may or may not be necessary. Such treatments may include (i) particle purification and/or (ii) particle activation. Particle activation may be necessary if the surface-displayed factor serves as a carrier for one or more bioactive factors (e.g. antibodies, proteins, small molecules, etc.). In this case, the bioactive factors would then be added directly to the fermentate or purified particles in order to allow their binding to the carrier protein displayed on the chimeric particle.

In most situations the founder and the trans-complementing genetic elements are introduced into the host cells by use of standard molecular biology methods such as transfection, infection and/or transformation as described by Ausubel et al. (ed.) “Current Protocols in Molecular Biology” John Wiley and Sons, 1995 (incorporated herein by reference). However in certain situations, it is contemplated that a particle producing host cell may be made by cell fusion. One such situation arises if it is found important to combine into one cell two phage genomes where each of which renders infected cells resistant against superinfection with the other phage genome. Although not considered a standard molecular biology methods Ausubel (1995) also gives detailed information regarding methods for cell fusion.

In order to ensure that the component encoded by the trans-complementing genetic element is directed to the surface of said particle during the assembly and/or release of the particle, this invention contemplates fusing the gene encoding a desired polypeptide (gene 1) to a second gene (gene 2) such that a fusion protein is generated during transcription of the trans-complementing genetic element. Gene 1 is typically a phage-encoded gene, and it is preferably a capsid protein from a phage that infects one or more lactic acid bacteria, or a portion thereof. Gene 2 (the “unrelated peptide sequence”) typically codes for antigens, allergens, virulence proteins, receptors, ligands etc. or a part thereof. Fusion of genes 1 and 2 may be accomplished by inserting gene 2 into a particular site on a plasmid that contains gene 1, or by inserting gene 1 into a particular site on a plasmid that contains gene 2 using standard molecular biology techniques such as those described in Ausubel, 1995 supra; and Sambrook, 1989 supra. Alternatively, the gene 2 may be fused to gene 1 by PCR using a technique called gene splicing by overlap extension (SOEing), as described by Horton, 1995.

In most embodiments of the invention the component that is directed to the surface of said particle during the assembly and/or release of the particle is a fusion protein that typically is a translational fusion between a sequence that codes for a peptide or protein that directs the fusion protein to the surface of the chimeric phage-derived particle and an unrelated peptide or protein coding sequence. The unrelated peptide sequence of said fusion protein in one particular preferred embodiment of the invention encodes an antigen and/or allergen able to elicit an immune response in humans and/or animals. The unrelated peptide sequence is in other embodiments of the invention selected from the group of peptide or protein sequences consisting of peptide or protein sequences that is contained in pathogenic (i.e. disease causing) peptides that are pathogenic to plants, humans, animals, fungi, or bacteria. In other embodiments of the invention, the unrelated peptides or protein sequence of the fusion protein is selected from the group of peptides. Whose interaction with plants, humans, animals, fungi, or bacteria, may be considered non-beneficial but not pathogenic, peptides that comprise part of a virulence factor, and peptides that enables the specific binding of at least one molecule.

As mentioned, it is the object of the present invention to provide a safe composition of chimeric phage-derived particles that can be administrated (e.g. by ingestion, injection, or topical application) to humans and/or animals without compromising the efficacy of the particles that display various factors. According to the invention, an additional amount of safety is obtained by selecting phages that are specific to bacterial host cells that are selected form the group of bacteria the use of which have been evaluated by the United States Food and Drug Administration, Center for Veterinary Medicine and/or analogous agency and approved as food additives of which enjoy GRAS status for use in food or feed. In one particular preferred embodiment of the invention it is contemplated that the bacterial host cell is regarded GRAS with respect to their use in dairy food products, but other GRAS categories are contemplated as well.

The European Food and Fed Cultures Association and International Dairy Federation (EFFCA/IDF) have in cooperation compiled an inventory of microorganisms with a documented safe history of use in food. The inventory is contained in full in the paper of G. Mogensen et al. (2002) Inventory of Microorganisms with a documented history of use in food. Bulletin of the International Dairy Federation, No. 377 page 10-19 (which is incorporated herein by reference). Consequently in an important embodiment of the invention that the bacterial host cell is selected form the group of bacteria consisting of bacteria, which according to European Food and Fed Cultures Association and International Dairy Federation (EFFCA/IDF) are micro organisms with a documented history of use in food and feed (including the use in direct-fed microbial products) without adverse effects. In particular bacteria selected from the group of lactic acid bacteria has a well documented history of safe use in food and feed products. In general, lactic acid bacteria can be described as gram-positive, catalase negative facultative anaerobic or microaerophilic bacterium that ferments sugars with the production of acids including lactic acid as the predominant fermentation product. The industrially most useful lactic acid bacteria are found among non-pathogenic Lactococcus spp., Streptococcus spp., Lactobacillus spp., Leuconostoc spp., Pediococcus spp., Brevibacterium spp., Enterococcus spp., and Propionibacterium spp. Additionally, lactic acid producing bacteria belonging to the group of the strict anaerobic bacteria, including bifidobacteria, i.e. Bifidobacterium spp. that are frequently used as food starter cultures or adjuncts alone or in combination with lactic acid bacteria, are generally included in the group of lactic add bacteria. Thus, a further important embodiment of the invention the bacterial host cell is selected from the group of non-pathogenic bacteria genera consisting of non-pathogenic Bifidobacterium spp., Brevibacterium spp., Enterobacter spp., Enterococcus spp., Lactobacillus spp., Lactococcus spp., Leuconostoc spp., Oenococcus spp., Pediococcus spp., Propionibactedium spp., Staphylococcus spp., and Streptococcus spp.

In Denmark all food cultures must be notified to the Danish Veterinary and Food Administration and their use accepted by the Administration before they can be used by the food industry. Table 1 mentions the bacterial species and subspecies the approved food cultures has been classified to.

TABLE 1
Classification of bacterial food cultures
accepted for use in food in Denmark

Arthrobacter globiformis
Bifidobacterium adolescentis
Bifidobacterium animalis
Bifidobacterium bifidum
Bifidobacterium breve
Bifidobacterium infantis
Bifidobacterium lactis
Bifidobacterium longum
Bifidobacterium pseudolongum
Bifidobacterium thermophilus
Brevibacterium casei
Brevibacterium linens
Corynebacterium flavescens
Enterococcus aerogenes
Enterococcus faecium
Hafnia alvei
Kocuria varians
Lactobaccillus delbrueckii subsp. lactis
Lactobacillus acidophilus
Lactobacillus alimentarius
Lactobacillus alimentarius brevis var. lindneri
Lactobacillus bavaricus
Lactobacillus brevis
Lactobacillus brevis var. lindneri
Lactobacillus bulgaricus
Lactobacillus carnis
Lactobacillus casei
Lactobacillus casei var. rhamnosus
Lactobacillus curvatus
Lactobacillus delbrueckii
Lactobacillus delbrueckii subsp. bulgaricus
Lactobacillus delbrueckii subsp. lactis
Lactobacillus farciminis
Lactobacillus helveticus
Lactobacillus jensenii
Lactobacillus lactis
Lactobacillus lactis subsp. lactis
Lactobacillus lactis subsp. lactis biov. diacetyllactis
Lactobacillus leichmanii
Lactobacillus paracasei paracasei
Lactobacillus paracasei subsp. paracasei
Lactobacillus pentosus
Lactobacillus plantarum
Lactobacillus rhamnosus
Lactobacillus sake
Lactobacillus sanfrancisco
Lactobacillus xylosus
Lactococcus lactis sub. lactis biovar. diacetylactis
Lactococcus acidophilus
Lactococcus lactis ssp. cremoris
Lactococcus lactis ssp. lactis
Lactococcus lactis subsp. cremoris
Lactococcus lactis subsp. lactis diacetylactis
Leuconostoc carnosum
Leuconostoc citrivorum
Leuconostoc dextranicum
Leuconostoc mesenteroides subsp. cremoris
Leuconostoc pseudomesenteroides
Micrococcus varians
Oenococcus oeni
Pediococcus acidilactici
Pediococcus pentosaceus
Propionbacterium Shermanii
Propionibacterium acidipropionici
Propionibacterium arabinosum
Propionibacterium freudenre ichii ssp. spermanii
Propionibacterium freudenreichii
Rhodosporidium infirmominiatum
Staphylococcus carnosus
Staphylococcus xylosus
Streptococcus cremoris
Streptococcus diacetylactis
Streptococcus durans
Streptococcus faecium
Streptococcus lactis
Streptococcus salivarius subsp. thermophilus
Streptococcus thermophilus

Such acceptance is a very strong indication that the bacteria can be considered safe, and consequently also phages that are specific for these safe “food grade” bacteria and thus inevitably infects such bacteria, generally can be regarded as safe. Therefore, in an important embodiment of the present invention the bacterial host cell is selected from the group of bacteria consisting of Arthrobacter globiformis, Bifidobacterium adolescentis, Bifidobacterium animalis (previously Bifidobacterium bifidum), Bifidobacterium breve, Bifidobacterium infantis, Bifidobacterium lactis, Bifidobacterium longum, Bifidobacterium pseudolongum, Bifidobacterium thermophilus, Brevibacterium casei, Brevibacterium linens, Corynebacterium flavescens, Enterococcus aerogenes, Enterococcus faecium, Hafnia alvei, Kocuria varians, Lactobaccillus delbrueckii subsp. lactis, Lactobacillus acidophilus, Lactobacillus alimentarius, Lactobacillus alimentarius brevis var. lindneri, Lactobacillus bavaricus, Lactobacillus brevis, Lactobacillus brevis var. lindneri, Lactobacillus bulgaricus, Lactobacillus camis, Lactobacillus casei subsp. casei, Lactobacillus casei var. rhamnosus, Lactobacillus cremoris, Lactobacillus curvatus, Lactobacillus delbrueckii, Lactobacillus delbrueckii subsp. bulgaricus, Lactobacillus delbrueckii subsp. lactis, Lactobacillus farciminis, Lactobacillus helveticus, Lactobacillusjensenii, Lactobacillus lactis, Lactobacillus lactis subsp. lactis, Lactobacillus lactis subsp. lactis biov. diacetylactis, Lactobacillus leichmanii, Lactobacillus paracasei (previously Lactobacillus casei), Lactobacillus paracasei paracasei, Lactobacillus paracasei subsp. paracasei, Lactobacillus pentosus, Lactobacillus plantarum, Lactobacillus rhamnosus, Lactobacillus sake (earlier L. alimentarius), Lactobacillus sanfrancisco, Lactobacillus xylosus, Lactocococcus lactis sub. lactis biovar. diacetylactis, Lactococcus (formerly Streptococcus) lactis subsp. cremoris, Lactococcus acidophilus, Lactococcus lactis, Lactococcus lactis spp. diacetilactis (previously Streptococcus diacetilactis), Lactococcus lactis subsp. cremoris, Lactococcus lactis subsp. lactis, Lactococcus lactis subsp. lactis diacetylactis, Leuconostoc camosum, Leuconostoc citrivorum, Leuconostoc dextranicum, Leuconostoc mesenteroides subsp. cremoris, Leuconostoc pseudomesenteroides, Micrococcus varians, Oenococcus oeni (previously Leuconostoc oenos), Pediococcus acidilactici, Pediococcus pentosaceus, Propionibacterium acidipropionici, Propionibacterium arabinosum, Propionibacterium freudenreichii ssp. spermanii, Propionibacterium freudenreichii, Propionibacterium shermanii, Rhodosporidium infirmominiatum, Staphylococcus camosus, Staphylococcus xylosus, Streptococcos salivarius subsp. thermophilus, Streptococcus cremoris, Streptococcus diacetylactis, Streptococcus durans, Streptococcus faecium, Streptococcus lactis and Streptococcus thermophilus (previously Streptococcus salivarius subsp. thermophilus).

From Table 1 and the discussion above it can be deduced that non-pathogenic microorganisms selected from the group of bacterial genera consisting of Arthrobacter spp., Bifidobacterium spp., Brevibacterium spp., Corynebacterium, Enterobacter spp., Enterococcus spp., Hafnia spp., Kocuria spp., Lactobacillus spp., Lactococcus spp., Leuconostoc spp., Micrococcus spp., Oenococcus spp., Pediococcus spp., Propionibacterium spp., Rhodosporidium spp., Staphylococcus spp. and Streptococcus spp. is very likely to obtain the approvement of an governmental body and thus forms a further important embodiment of the invention.

Several other non-pathogenic microorganisms, including Bacillus spp., especially Bacillus coagulans, Bacillus lentus, Bacillus licheniformis, Bacillus pumilus, and Bacillus subtillis have also been approved by the United States Food and Drug Administration, Center for Veterinary Medicine and/or analogous agency and found to present no safety concerns when used as direct-fed microbial products for use in animal feed. Thus, a further important embodiment of the invention the bacterial host cell is selected from the group of non-pathogenic bacteria consisting of non-pathogenic Bacillus coagulans, Bacillus lentus, Bacillus licheniformis, Bacillus pumilus, and Bacillus subtillis.

In order for a GMO to be used in food, it must fulfill a number of safety criteria and must be able to attain GRAS status in the United States. While there is no official definition of what constitutes a ‘food-grade’ GMO, a working definition has been elaborated (Johansen, (1999) Genetic engineering (b) Modification of bacteria. In: Encyclopedia of Food Microbiology (Robinson, R., Batt, C. and Patel, P., eds). Academic Press, London, pp. 917-921) and at least one Lactococcus strain fulfilling this definition has been affirmed as GRAS and brought to the market in the United States. An important element of this definition is that a food-grade GMO can only contain DNA from the same species. In a broader definition of food-grade, genes from other GRAS food microorganisms would be considered acceptable (Johansen, 1999). In either case, the use of antibiotic resistance genes as selectable markers is not allowed. Many of the strains constructed at universities and in research institutions are for ‘proof of concept’ and as such there is often no need for them to be food-grade. Thus, antibiotic resistance markers have been used due to the ease of working with them. If these strains are to be used in the industry, it is necessary to eliminate all inappropriate DNA or to reconstruct the strains in a more appropriate manner. Although a virus such as the particles of the present invention that do not contain any genetically manipulated genetic material and only contains what with all due respect must be considered genetic material of its own species hardly qualify to be classified as an GMO, the reality is that the discussion regarding the safety of GMOs remain and consequently an interesting embodiment of the present invention is a method of producing chimeric particles wherein the bacterial host cell prior to the addition of the said two or more genetic elements can be regarded as food-grade microorganism or food-grade GMO as defined by Johansen (1999).

Use of these safe microorganisms ensures that toxic factors (e.g. superantigens, endotoxins, lipopolysaccharides, etc.) that might be intrinsic to other potentially non-safe microorganisms (e.g. Escherichia coli) are not present in the final preparation of chimeric phages-derived particles (since they are not associated with these safe microorganism). This distinction represents a significant improvement over the prior art that typically describes the use of potentially non-safe microorganisms to produce similar products, such as virus-like particles.

Typically, the genomes of naturally occurring phages code for one or more components that act alone or in concert with other phage or bacterial encoded factors to induce lysis of the bacterial host cell. In one embodiment of the present invention, the particles are released from said bacterial host cells by the action of phage-genome encoded component or components (“phage encoded lysis components”). In a preferred embodiment the phage encoded lysis components are encoded by the founder genetic element, but embodiments wherein the at least one of the phage encoded lysis components are coded by a different genetic element is also envisioned. One such situation occurs when at least one of the phage encoded lysis components are encoded by a phage or pro-phage genome that is different from the founder genetic element or even encoded by one or more trans-complementing genetic elements.

While a number of different factors have been associated with phage-induced lysis of bacteria a important embodiment of the invention is a method of production wherein the phage-genome encoded component or components comprise holin and/or endolysin and/or lysozyme.

A preferred use of the method of production of a composition comprising chimeric phage-derived particles is to provide a composition for human or animal intake that comprise the chimeric particles of the invention. One example of such a composition is a particle-containing fermented dairy food product such as a yogurt. In the case of such fermented food products, the culture of bacterial host cells that are subjected to conditions which results in formation of the chimeric particles could simply be the same bacterial culture that are also performing the fermentation of the milk product. Since the bacteria/virus system could be considered “safe food grade” organisms, and thus safe for human intake, such particle containing yogurt could be brought to the market while claiming the additional benefits that is associated with the additional surface displayed factors of the chimeric particle.

While it is not required to further purify or isolate the chimeric particles from such culture of bacterial host cells in order to obtain a fermented composition that can be brought to the market, for certain applications a purification or isolation step may be required for other uses of the chimeric particles.

One example of a use which appear to require that the chimeric particles are—at least to some degree—isolated or purified from the culture of bacterial host cells is the use of a chimeric phage derived particle (such as a chimeric phage, chimeric phage-like or chimeric phage ghost particle) to produce a vaccine. Accordingly the present invention also provide a method for obtaining a chimeric phage-derived particle that comprise at least two different surface displayed proteins”, said method comprising the steps of: (i) obtaining a composition from where said chimeric phage-derived particles may be isolated as already described, and (ii) isolate the chimeric phage-derived particles from the composition.

The terms “isolated” or “purified” refer to a composition of chimeric phage-derived particles that is significantly or considerably free from unwanted components that normally accompany the particles in their native state (i.e. components of the particle-producing bacterial culture that is not the chimeric particles such as e.g. bacteria, bacterial debris and growth medium constituents). Particularly, it means that at least 50% of the unwanted components have been removed from the composition, more preferably that at least 75% have been removed, and most preferably that at least 99% unwanted components have been removed from the composition.

The art describes a number of methods that can be applied to isolate phage particles. Since the particles of the present invention in many aspects appear as chimeric phage-like particles many of these methods can be applied to isolate the chimeric phage-derived particles from the composition. It is contemplated that the particles can be isolated or purified by standard methods for phage purification such as centrifugation (including CsCl density centrifugation), polyethylene glycol (PEG) precipitation, and affinity chromatography. Detailed description of these and other suitable methods for isolation can be found in Ausubel et al. (ed.) “Current Protocols in Molecular Biology”. John Wiley and Sons, 1995; and Sambrook, J. et al., Molecular Cloning: A Laboratory Manual. Cold Spring Harbor Laboratory Press, NY, Vol. 1,2,3, 1989. Both of which are incorporated herein by reference. Chimeric particles can also be purified using separation methods based upon the intrinsic physical properties of the said particles, such as (but not limited to) micro- and nano-filtration, molecular size exclusion, and isoelectric focusing.

As described herein, the invention provides a method to produce a chimeric phage-derived particle (such as a chimeric phage, chimeric phage-like or chimeric phage ghost particle) that in addition to at least one normal phage component display or comprise at least one additional component and wherein the at least one normal phage component is coded by a genetic element that comprises a substantial part of a phage genome (the founder genetic element) and the at least one additional component being coded by a different genetic element, (the trans-complementing genetic element). An essential feature of the particles of the invention is that they do not comprise the sequence encoding for at least part of the additional component.

Whereas the trans-complementing genetic element will only code for one or a few of the components of the chimeric particle the founder genetic element typically will comprise a substantial part of a phage genome coding for a number of phage proteins. As illustrated in FIG. 1, the three types of chimeric particles will typically comprise several normal phage components in addition to the at least one additional component. In most situations, such particles will have a size and appearance that resembles the naturally occurring phage isolate from where the founder genetic element was derived. In the present context, the naturally occurring phage isolate from where the founder genetic element was derived is referred to as the “founder particle”.

The chimeric particles of the invention may be comprised solely by phage components, for example they may be composed primarily of normal phage components coded by the founder genetic element and one or a few phage components originating from a complete unrelated virus being expressed from the trans-complementing genetic element. Such a situation arises if the present invention is used to produce chimeric particles expressing a phage encoded virulence factor. In another embodiment of the present invention, the particle in addition to several normal phage components displays at least one additional component that may not necessarily be a normal phage component. Principally, the virus/host cell systems described by the present invention may comprise any of a wide range of components that will associate with or bind to the particles formed and whereby the genes on trans-complementing genetic element fail to be incorporated into the chimeric particle. However in a preferred embodiment the at least one additional component coded by the trans-complementing genetic element is a protein. In particular an embodiment wherein the at least one additional component that is coded by the trans-complementing genetic element is a fusion protein being a fusion between a peptide sequence that direct the fusion protein to the surface of said chimeric phage-derived particle and an unrelated peptide sequence is preferred.

A number of proteins have been described that are directed to the surface of phages during phage assembly and release. One category of such proteins is the phage capsid proteins which is the proteins that form the coat or capsid of a phage. When a peptide that comprise a “functional part”, defined here as the part of a phage capsid protein that direct the protein to the capsid, is fused to a another peptide of interest, then the fusion peptide will be directed to the capsid. The result being that the peptide of interest, e.g. an antigen or epitope of interest, are directed to the phage coat and displayed on the surface of the coat of the chimeric particle. Accordingly in one embodiment of the present invention the fusion protein comprise a peptide sequence comprising a functional part of a phage capsid protein. In addition to capsid proteins, which are an integral part of all phages, phages may comprise other surface structures. In analogy with the capsid proteins also the proteins that form these other structures contains signals (peptide sequences) that direct the proteins to the surface of the phages. Examples of such surfaced displayed phage proteins are the proteins that form a phage collar, a phage whisker, a phage tail, a phage base plate, and/or a phage tail fiber the use of such proteins to direct a fusion protein to the surface of the phage is also contemplated.

As previously discussed, the emphasis of the present invention is to provide chimeric particles, which to a large extent is based on virus/host cell systems being generally recognized as safe. One example of a group of such generally recognized, as safe organisms are the lactic acid bacteria. Some of the industrially most useful lactic acid bacteria are found among bacteria of the Lactococcus species (spp.). In real life industrial settings phage infection occur at irregular intervals although great care normally is taken to avoid the phage infection. Thus, in one preferred embodiment of the invention a virus/host cell system that comprises a lactococcal species and its related phages is preferred. In one preferred embodiment phages similar to the lactococcal type phage c6A are preferred. One particular example of such phages is phage c2. This group of phages is quite complex phages and a number of phage proteins may be utilized to construct fusion proteins that will be directed to the surface of the phage. Accordingly, in one preferred embodiment of the present invention the fusion protein comprise a peptide sequence comprising a functional part of a phage capsid protein selected from the group of phage proteins consisting of gpL1, gpL2, gpL3, gpL4, gpL5, gpL6, gpL7, gpL8, gpL9, gpL10, gpL11, gpL12, gpL13, gpL14, gpL15, gpL16, and gpL17 derived from a phage similar or identical to the lactococcal type phage c6A phages, such as e.g. phage c2.

Although a phage's genome may by introduced into a cell by a number of methods, usually the most efficient way to introduce the genome into the cytoplasm of host bacterium is by infection. Thus, in another preferred embodiment, the invention relates to an infective chimeric phage or a chimeric phage-like particle. Since the chimeric phage ghost particles of the present invention does not contain genetic material and consequently not able to introduce their genome into host cells they are not considered infective. They may however typically express a “host-cell specificity” similar to the founder particle and may accordingly adhere to specific bacteria and perform several of the actions that are characteristic for the early phases of infection. Thus, an embodiment of the present invention is a chimeric phage-derived particle (eg a chimeric phage, chimeric phage-like or chimeric phage ghost particle) that with respect to the infective chimeric phages or a chimeric phage-like particles are infective or with respect to the chimeric phage ghost particles are able to adhere to specific bacteria and perform several of the actions that are characteristic for the early phases of infection and that exhibits a host specificity that is determined by the founder genetic element.

In most embodiments of the present invention, the chimeric phage-derived particles retain a host specificity that is identical to the naturally occurring phage isolate from where the founder genetic element was derived (i.e. the “founder particle”). Whereas the host cell specificity thus in most settings are determined by the founder genetic element it is contemplated that the trans-complementing genetic element as well could code for components that determined the host cell range or host specificity. Thus, also a chimeric phage-derived particle wherein the host specificity is altered relative to the naturally occurring phage isolate is contemplated. Similarly either the founder genetic element, the trans-complementing genetic element(s) or even both type of elements may code for factors or comprise variations of genes that results in a chimeric phage-derived particle that exhibits an increased or a reduced or even lost capacity to infect bacteria relative to the naturally occurring phage isolate from where the founder genetic element was derived.

Examples of particles, which according to the definition herein are not infectious, are chimeric chimeric phage-like or chimeric phage ghost particles, since in both cases the chimeric particles do not contain any genetic material.

As previously mentioned the trans-complementing genetic element codes for a fusion protein in more preferred embodiments of the present invention. Typically, this fusion protein is directed to the surface because it comprises part of phage capsid protein, which comprises a localization signal ensuring that the factor is displayed on the surface of the particle. However, a factor coded by the trans-complementing genetic element may also be directed to the surface of the phage due to other mechanisms. In one embodiment of the invention the fusion protein is able to associate with virus-encoded components and will be directed to the surface of the chimeric phage during its assembly in and release from the host cells because of this association. In another embodiment the fusion protein contain peptide sequences that are able to associate with virus-encoded components in addition to part of phage capsid protein that comprise a particle localization signal. The virus-encoded components that the fusion protein is able to associate with may be any virus-encoded components including components of a virus different from the founder particle as well as virus-encoded proteins comprised in the naturally occurring phage isolate from where said substantial part of a phage genome was derived (the founder particle). Likewise the fusion proteins are contemplated to be constructed that are able to associate with said virus-encoded one or more proteins of the founder particle isolate prior to lysis of the bacterial host cell as well as after the chimeric particles are released form the host cell.

The fusion protein may furthermore be part of so-called “binding partners”. “Binding partners” are substances that specifically bind to one another, usually through noncovalent interactions. Examples of binding partners include ligand-receptor, streptavidin-Biotin, polyhistidine-Ni chelate, antibody-antigen, drug-target, and enzyme-substrate interactions. Binding partners are extremely useful in both therapeutic and diagnostic fields.

In order to avoid a situation hereby a fusion protein must compete with the wild-type phage-encoded capsid protein for “open” or free sites in the chimeric phages, one may use phage genomes that carry a “destructive” mutation in the gene coding for the particular capsid protein as founder genetic element. Examples of such destructive mutations are missense mutations and deletions.

Preferred embodiments of the invention is based on relatively complex types of phages that comprise more capsid proteins and that in some embodiments in addition also comprise proteins of such structures as phage collar, phage whisker, phage tail, phage base plate, and/or a phage tail fibers. In principle there is no strict limit to the number of fusion proteins the trans-complementing genetic element or elements may code for. Thus, in another embodiment, the present invention relates to a particle that in addition to the at least one normal phage component comprise at least two additional components that are not encoded by the founder genetic element.

The unrelated peptide sequence of the fusion protein corresponds to the additional component of the fusion protein (i.e. the non-virus part in most embodiments) and may in principle be any sequence. In preferred embodiments, however, the unrelated peptide sequence is derived from the genome of plants, humans, animals, fungi, bacteria, or viruses or may be a synthetic or randomly generated amino acid sequence. In particular, the sequence may be derived from a pathogen of plants, humans, animals, fungi, or bacteria. In preferred embodiments the unrelated peptide sequence is derived from a microorganism whose interaction with plants, humans, animals, fungi, or bacteria, may be pathogenic (i.e. disease-producing). In further preferred embodiments the unrelated peptide sequence is derived from a virulence factor comprised of a specific sequence of amino acids or a portion thereof. The term “a portion” corresponds to an amino acid sequence of a length that as a minimum allow specific antibodies to be raised against said virulence factor. Normally such portion of a peptide constitutes what is referred to as an antigenic determinant. An “epitope” refers to an antigenic determinant of a polypeptide. An epitope can comprise as few as 3 amino acids in a spatial conformation that is unique to the epitope. Generally, an epitope consists of at least 6 such amino acids, and more usually at least 8-10 such amino acids.

Although the microorganisms from which the unrelated peptide sequence was isolated may still be considered non-beneficial even if they do not cause disease. Examples of such non-beneficial microorganisms are microorganisms that decreases the feed efficiency, e.g. by reducing the uptake of nutrients from the feed, but do not cause any disease. In further embodiments the unrelated peptide sequence of said fusion protein is derived from a microorganism whose interaction with plants, humans, animals, fungi, or bacteria, may be considered non-beneficial but not pathogenic.

An interesting application of the present invention is to use the technology to extend the retention time of phage particles in the gastrointestinal tract during phage therapy. For example, a mucin-binding protein may be expressed on the coat of the phage. This protein would act to anchor the phage in the gut, while allowing it to specifically infect (via its free tail) its pathogenic target microbe. Alternatively, the phage could be coat tagged with a protein that facilitates binding to a probiotic bacteria strain. This would allow the chimeric particle and the probiotic bacteria to be easily co-administered and lead to a prolonged retention-time of the probiotic bacterium in the gut. Accordingly, in an important embodiment of the present invention, the unrelated peptide sequence of said fusion protein encodes a protein or peptide that facilitates and/or enables the binding of the chimeric phage-derived particle to receptors found on a solid surface, a biofilm, human or animal cells, or other microbes.

The unrelated peptide sequence of the fusion protein may as well comprise a protein or peptide (e.g. polyhistidine) that facilitates and/or enables the conditional binding of the chimeric phage, phage-like, or phage-ghost particle to a matrix (e.g. nickel-nitrilotriacetic acid (Ni-NTA) metal-affinity chromatography matrices). Such peptide sequences can be used for the purification of said chimeric phages-derived particles. They may in addition be used as a tag (or epitope) that could be used in immunization.

In a most preferred embodiment of the present invention the unrelated peptide sequence of the fusion protein encodes an antigen and/or allergen able to elicit an immune response in humans and/or animals.

Furthermore the fusion protein may also be part of a binding partner pair. A number of examples from prior art show that binding partners are extremely useful in both therapeutic and diagnostic fields, thus in one embodiment of the present invention the unrelated peptide sequence comprise a peptide that enables the specific binding of at least one molecule, in particular the situation wherein the fusion protein functions as an extracellular receptor is contemplated. Wide ranges of molecules that potentially may bind to such a fusion protein are envisioned. In particular molecules of biological origin such as a protein, lipoprotein, glycoprotein, carbohydrate or a lipid is relevant, but also molecules such as various metals (e.g. Cd, Ni, Fe) and certain organic molecules of non-biological origin (e.g. certain pesticides or their degradation products) are contemplated.

In certain cases the actual binding of toxin to various particles or large molecular structures lead to the inactivation or neutralization of the toxin. Toxins may also be displaced from a solution or suspension by adding toxin binding particles to the solution or suspension, allowing the toxin to bind to the particle and remove the particle from the solution or suspension e.g. by centrifugation. Thus, one additional embodiment of the present invention is the use of the chimeric particles for binding and/or neutralization of biological toxins. As previously mentioned it is fully within he scope of the invention to provide chimeric particles unto which two or more different fusion proteins are displayed. It is contemplated that such chimeric particles that display two or more specific binding affinities, here referred to as “additional tags”, will have wide uses. Examples of useful additional tags are the binding partners previously described. Such particles would be useful for removing toxins from a solution or suspension if for instance the one additional tag is member of a binding partner pair such as poly-his and the other tag binds specifically to the toxin, then such a particle could conveniently be removed by affinity chromatography, thus resulting in the removal of the toxin. Similarly the additional tags could be used for purification or isolation of the chimeric particle. Consequently a further interesting embodiment of the present invention is a composition comprising a chimeric phage-derived particle that displays an additional tag that facilitates their binding and/or downstream purification.

In other embodiment, the present invention relates to a method for the production of a pharmaceutical composition comprising the steps of the method of the present invention and further the step of formulating at least one of said chimeric particles in a pharmaceutically acceptable form.

If chimeric phage particles according to the invention are administered to an animal, they will act as a vaccine by delivering specific antigens to the immune system. In addition, they could be specifically tagged to target T- or B-lymphocytes to deliver their antigenic cargo. Unlike live attenuated vaccines or traditional inactivated vaccines, there is no possibility for horizontal transfer of the virulence genes to the indigenous flora of the host. Further, there is no possibility for the tagged phages to revert to a disease-causing variation, which may occur in attenuated or live vaccines. Thus, in the most preferred embodiment the chimeric phage-derived particle is used to produce a vaccine. In particular a vaccine that can be administered to mucosal surfaces, including the conjunctiva, the gastrointestinal tract, the respiratory tract, and the urogenital tract of humans and/or animals is contemplated, and in particular a vaccine that is based on Lactococcus lactis is preferred. In certain instances it has been found that antigen containing compositions can be used to treat allergy. Consequently, yet an important embodiment of the invention is the use of a composition comprising a chimeric phage-derived particle for the treatment of allergies.

Multiple tagged particles can be used to target and kill cancer or pathogens cells. For exampie, one tag may be specific for a cancerous cell or a pathogen, while a second tag can encode a toxin. It may be possible for the cytotoxin to be sequestered from the host until after it has bound a cancer cell. Thus, these particles could serve as a platform for targeted killing.

Furthermore it is contemplated that the chimeric particles can be tagged with proteins that allow them to specifically bind to and coat or cover a pathogen. If the pathogen is required to bind to certain receptors in its host in order to function as a pathogen (e.g. in the gut), then pathogen could be effectively neutralized. This appears an interesting application in particular in relation to functional food.

Hence, in another most preferred embodiment of the invention, the chimeric phage-derived particle is used to produce compositions that competitively exclude pathogens or non-desirable microorganisms.

The particles of the present invention are contemplated to be especially efficacious with respect to produce a composition, which competitively excludes pathogens or non-desirable microorganisms associated with mucosal surfaces, including the conjunctiva, the gastrointestinal tract, the respiratory tract, and the urogenital tract of humans and/or animals.

It is further contemplated that the particles will find wide use and even allowing the production of a composition, which competitively excludes pathogens or non-desirable microorganisms, associated with plants and/or weeds relevant to human agriculture.

Probiotics constitute a class of microorganisms defined as live microbial organisms that beneficially affect animal or human hosts. The beneficial effects include improvement of the microbial balance of the intestinal micro flora and the improvement of the properties of the indigenous microflora. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts. Probiotics may suppress undesired intestinal organisms by producing antibacterial compounds and/or by successful competition for nutrients and/or adhesion sites in the gastrointestinal tract. Additionally, they may alter microbial metabolism by increasing or decreasing enzyme activity or they may stimulate the immune system by increasing antibody levels or increasing macrophage activity. Probiotics may even express anti-tumor activity or facilitate lowering of blood cholesterol levels, Fuller (1989); Elmer (2001)).

Probiotic microorganisms have been identified among microorganisms classified as yeasts, fungi and bacteria. An important aspect of a probiotic agent is that it should relate to live microorganisms. In the present context phage and phage-like particles that may infect host cells is considered as live microorganisms. As described previously and discussed in the examples, chimeric particles of the present invention may antagonize specific groups of undesired organisms, suppress undesired intestinal organisms, stimulate the immune system and in many ways confer a health benefit on the host when administered in adequate amounts. Accordingly, the particles of the present invention qualify to be referred to as probiotic microorganisms or probiotic agents. Consequently, in a preferred embodiment of the present invention, the chimeric phage-derived particles are used to produce a probiotic composition. Whereas chimeric phage ghost particles not are considered as living organisms in the present context, chimeric phage ghost particles may find important use in the manufacture of a probiotic composition as a supplement to probiotic organisms, thus providing the probiotic composition with certain additional advantages.

In another embodiment the particles are used to produce a direct-fed microbial composition. Such a direct-fed microbial composition may in addition to the chimeric particles of the present invention also comprise probiotic bacteria.

Phage therapy relates primarily to the utilization of phages to eliminate and/or reduce the number of pathogenic and/or otherwise non-desirable microorganisms. In further embodiments the present invention describes a composition comprising a chimeric phage-derived particle according to any of the preceding claims that is useful for phage therapy. Such compositions will, in many instances, appear as a pharmaceutical composition, which in addition to the chimeric particles is formulated with a pharmaceutically acceptable carrier and/or diluents. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. A review of conventional formulation techniques can be found in e.g. “The Theory and Practice of Industrial Pharmacy” (Ed. Lachman L. et al, 1986) or Laulund (1994), which are included herein by reference. These pharmaceutical compositions can be administered to the subject at a suitable dose. Administration of the suitable compositions may be effected by different ways, e.g., by intravenous, intraperitoneal, subcutaneous, intramuscularly, topical, intradermal, intranasal or intrabronchial administration. The attending physician and clinical factors will determine the dosage regimen. As is well known in the medical arts, dosages for any one patient depends upon many factors, including the patient's size, body surface area, age, the particular compound to be administered, sex, time and route of administration, general health, and other drugs being administered concurrently.

The chimeric particles of the present invention may also find non-therapeutic use to reduce or otherwise control the number of pathogenic and/or otherwise non-desirable microorganisms present in a defined environment. Thus, in another embodiment of the present invention, the chimeric phage-derived particles are used as biocontrol agents specific for pathogenic and/or non-desirable microorganisms.

The chimeric particles of the invention may be engineered so that they express a specific binding affinity to one or more cytotoxic agents and can thus be used as carriers of said cytotoxic agent. A special case is the situation wherein the cytotoxic agent is a proteinaceous molecule that is translationally fused to a functional form of a phage (capsid) protein, thus a further embodiment of the invention is a composition comprising a chimeric phage-derived particle that is useful to neutralize, kill and/or impede, a pathogen or non-desirable microorganism by means other than those associated with conventional phage therapy or phage biocontrol through the delivery of one or more cytotoxic agent(s).

In a further embodiment a composition comprising a chimeric phage-derived particle is contemplated that is useful to neutralize, kill and/or impede, a pathogen or non-desirable microorganism by means other than those associated with conventional phage therapy or phage biocontrol by precluding the pathogen or non-desirable microorganism from associations that normally allow for the deleterious characteristics in vivo.

### LEGENDS

FIG. 1. Conceptual drawings of a native phage particle (1), chimeric phage particle (2), chimeric phage ghost particle (3) and chimeric phage-like particle (4). Nucleic acids (DNA or RNA) are depicted as a circle and indicated by arrow “a”. The surface displayed factor(s) are depicted as a small spheres indicated with arrow “b”.

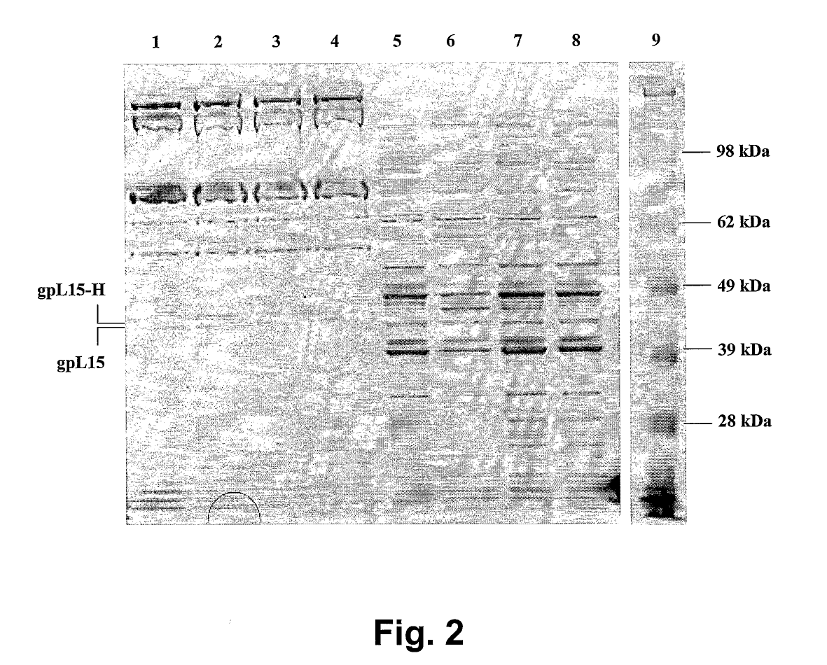

FIG. 2. Photo of SDS-PAGE gel showing that phage particles with chimeric gpL15 protein (ie gpL15-H) are obtained. Lane 1, φc2 propagated on MG1363 (control); Lane 2, φc2 propagated on MG1363 (pJMS245::I15) (control); Lane 3, φc2 propagated on MG1363 (pJMS245::115-H) (Isolate 1); Lane 4, φc2 propagated on MG1363 (pJMS245::I15-H) (Isolate 2); Lane 5, total proteins extracted from MG1363 (control); Lane 6, total proteins extracted from MG1363 (pJMS245::I15) (control); Lane 7, total proteins extracted from MG1363 (pJMS245::I15-H) (Isolate 1); Lane 8, total proteins extracted from MG1363 (pJMS245::I15-H) (Isolate 2); Lane 9, SeeBlue2 Protein Standard (Invitrogen, Carlsbad, Calif.).

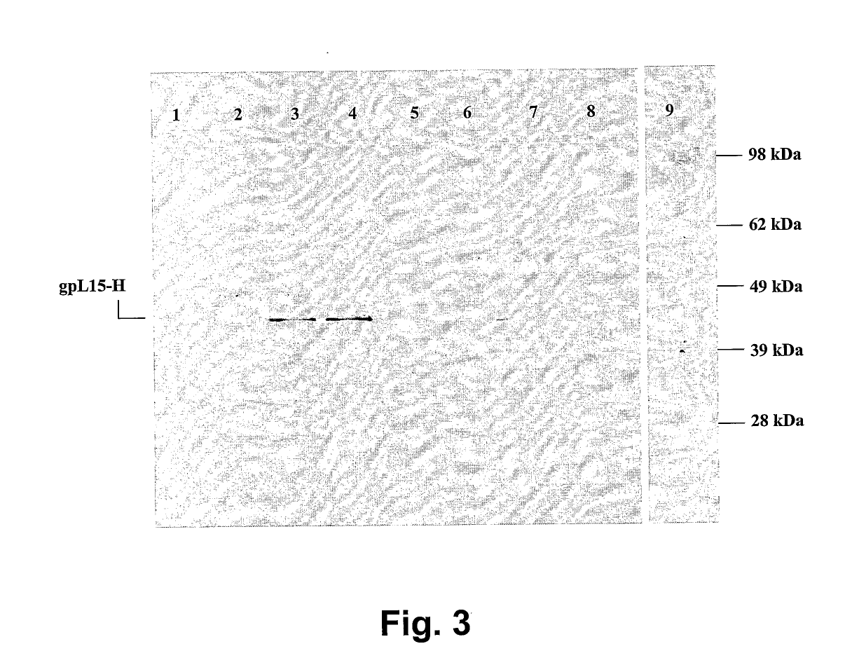

FIG. 3. Photo of western blot showing that chimeric phage particles comprising gpL15-H are obtained. Lane 1, φc2 propagated on MG1363 (control); Lane 2, φc2 propagated on MG1363 (pJMS245::I15) (control); Lane 3, φc2 propagated on MG1363 (pJMS245::I15-H) (Isolate 1); Lane 4, φc2 propagated on MG1363 (pJMS245::I15-H) (Isolate 2); Lane 5, total proteins extracted from MG1363 (control); Lane 6, total proteins extracted from MG1363 (pJMS245::I15) (control); Lane 7, total proteins extracted from MG1363 (pJMS245::I15-H) (Isolate 1); Lane 8, total proteins extracted from MG1363 (pJMS245::I15-H) (Isolate 2); Lane 9, SeeBlue2 Protein Standard (Invitrogen, Carlsbad, Calif.).

### EXAMPLES

### Example 1

### Construction of a Host Cells Suitable for the Constitutive Production of Chimeric Particles

Bacterial strains and growth conditions. All microbiological media were purchased from Becton, Dickinson & Company (Sparks, Md.). Unless otherwise indicated, all other reagents were of analytical grade and purchased from Sigma-Aldrich (St. Louis, Mo.). Escherichia coli strain MC1061 (Huynh et al., 1985) and One Shot® Top10 (Invitrogen, Carlsbad, Calif.) were propagated at 37° C. with aeration in Luria-Bertani broth. Lactococcus lactis subsp. cremoris strain MG1363 (Gasson, 1983) and derivatives thereof were propagated aerobically at 30° C. in M17 broth supplemented with 0.5% (w/v) glucose (M17-G). Phage c2 (φc2) and chimeric derivatives thereof were propagated in M17-G supplemented with 10 mM CaCl₂ (M17-GC) at 30° C. on derivatives of MG1363. For the selection of recombinant E. coli, chloramphenicol (5 μg/mL) or erythromycin (100 μg/mL) were added to media, as appropriate. For the selection of recombinant L. lactis, chloramphenicol (5 μg/mL) or erythromycin (5 μg/mL) were added to media, as appropriate. For solid media, agar was added at a final concentration of 1.5% (w/v) for base agar and 0.75% (w/v) for top agar. Bacterial stocks were maintained at −70° C. in fresh culture medium containing 15% (v/v) glycerol.

Purification of nucleic acids. Small-scale preparations of plasmid DNA were isolated from E. coli and L. lactis as described by Sambrook et al. (1982) and O'Sullivan and Klaenhammer (1993), respectively. Large-scale preparations of plasmid DNA were isolated using the Plasmid Midi Kit (Qiagen, Chatsworth, Calif.) according to the manufacturer's instructions. As appropriate, DNAs were extracted from agarose gels or enzymatic reactions using the QIAquick Gel Extraction Kit (Qiagen) or PCR Purification Kit (Qiagen), respectively. L. lactis φc2 genomic DNA was prepared using the Lambda Kit (Qiagen) according the manufacture's instructions. DNA ligations were purified and concentrated prior to electroporation using the MinElute PCR Purification Kit (Qiagen).

Polymerase chain reaction (PCR) and recombinant DNA techniques. PCR reactions were performed using an iCycler thermal cycler (Bio-Rad Laboratories, Hercules, Calif.) using TripleMaster DNA Polymerase Mix (Eppendorf, Hamburg, Germany) or Ex Taq™ Polymerase (TaKaRa, Shiga, Japan). DNA oligonucleotide primers were synthesized by Invitrogen, Inc. (Carlsbad, Calif.). When appropriate, restriction endonuclease recognition sites were incorporated into the 5′ end of DNA primers to facilitate the cloning of PCR products. Ligation reactions were performed using T4 DNA ligase (Invitrogen, Carlsbad, Calif.) according to the manufacturer's instructions. When appropriate, calf intestinal alkaline phosphatase (Promega, Madison, Wis.) was used to facilitate the cloning of PCR products. DNA sequencing reactions were performed by Northwoods DNA, Inc. (Solway, Minn.) and DNA sequences were analyzed using Lasergene v5.0 (DNAstar, Inc., Madison, Wis.) or Clone Manager version 6.0 (Scientific and Educational Software, Durham, N.C.).

Bacterial transformation. All electroporations were performed using a Bio-Rad Gene Pulser (Bio-Rad Laboratories, Hercules, Calif.) apparatus configured to 25 μF, 2.5 kV, and 200Ω. Preparation of electrocompetent E. coli MC1061 was conducted as described by Sambrook et al. (1982); electrocompetent L. lactis MG1363 was prepared utilizing the method described by Holo and Nes (1989).

Gene Product L15 (gpL15-H) expression system. In this study, gpL15 was used as a carrier for the incorporation and display of model antigens on the surface of the φc2 capsid, although the use of other peptides and proteins is also possible. This 43.2 kDa protein is known to be a minor structural component of the φc2 capsid (Lubbers et al., 1995) and is involved in host cell recognition for prolate-headed phages (Stuer-Lauridsen et al., 2003). Primers JS16_F-gpL15 and JS36_R-gpL15-H were used to amplify I15-H (SEQ ID No. 1), a recombinant version of the L. lactis φc2 I15 gene. I15-H encodes gpL15-H, a translational fusion protein that displays six contiguous histidine residues (hexahistidine) at its carboxy-terminus (SEQ ID No. 2). As a control, primers JS16_F-gpL15 and JS17_R-gpL15 were used to amplify the wild-type (unlabeled) I15 gene from the genome of L. lactis φc2 (control). BamHI restriction endonuclease recognition sites were incorporated into the 5′ ends of primers JS16_F-gpL15, JS17_R-gpL15, and JS36_R-gpL15-H. The resultant PCR products were restricted with BamHI and independently ligated to BamHI restricted pJMS245 (=pTRK687, Sturino (2002)), which encodes chloramphenicol resistance as a selectable marker. The resulting plasmid DNAs were purified and electroporated into electocompetent E. coli. Chloramphenicol resistant colony forming units (CFUs) were screened for the presence of insert and the orientation of the inserted gene was determined by restriction analysis and confirmed by DNA sequencing. Plasmids pJMS245::I15 and pJMS245::I15-H containing the inserts in the sense orientation were independently electroporated into MG1363. Chloramphenicol resistant CFUs were screened for the presence of either pJMS245::I15 or pJMS245::I15-H.

Primers Used:

JSI6_F-gpL15 (SEQ ID No. 3)
5′-CGCGGATCCAGATCTCACAATAGAAAGGGTATATAAATG-3′
JS17_R-gpL15 (SEQ ID No. 4)
5′-CGCGGATCCAGATCTCTATCCATTGTGTAGCCCTC-3′
JS36_R-gpL15-H (SEQ ID No. 5)
5′-CGCGGATCCCCCGGGCTAATGATGATGATGATGATGTCCATTGTGTA
GCCCTCTCATTCC-3′

### Example 2

### Production of Chimeric Phages

Precipitation and visualization of bacteriophages by SDS-PAGE. Mid-log cultures of L. lactis MG1363, MG1363 (pJMS245::I15) and two independent isolates of MG1363 (pJMS245::I15-H) were independently infected with wild-type 4c2 at a multiplicity of infection (MOI) of 0.1. The lytic infections were allowed to proceed until the culture was lysed completely. 500 mL phages lysates titered at >5×10⁹ plaque-forming units (PFU) per mL were concentrated by polyethylene glycol (PEG) precipitation according to the method described by Sambrook et al. (1982). The concentrated phage lysates were subjected to SDS-PAGE under reducing conditions using Novex 4-12% Bis-Tris Gels (Invitrogen, Carlsbad, Calif.) in 1×MOPS buffer as described by the manufacturer. As a control, total proteins were also isolated from uninfected control cultures via bead beating.

FIG. 2 illustrates typical results obtained by SDS-PAGE. Total proteins were isolated from uninfected control cultures via bead beating (Lanes 5-8) and compared to PEG-precipitated wild-type φc2 (Lanes 1-2) and chimerically labeled φc2 (Lanes 3-4). The gpL15 protein in Lanes 1-4 migrates as a 42-kDa protein, which is congruent with prior observations (Lubbers et al., 1995). As expected, only phage particles that were propagated on MG1363 (pJMS245::I15-H) were found to contain the 42+-kDa gpL15-H protein, which was produced in trans by the host-encoded plasmid, in addition to the wild type (phage-encoded) gpL15 protein. In contrast, the gpL15-H protein was not expressed at high enough levels to be visualized in total protein extracts isolated from two independent isolates of MG1363 (pJMS245::I15-H) grown in the absence of phage infection (Lanes 7-8). The gpL15-H protein is clearly visible in Lanes 3-4, however, indicating that the gpL15-H protein is (i) expressed by MG1363 (pJMS245::I15-H) and (ii) incorporated into phage particles at great efficiency when expressed by the host in trans. Further, as the intensity of the gpL15-H band in Lanes 3-4 nearly matches those of the wild-type gpL15 protein shown in lanes Lane 1-2, indicating that the majority of the wild-type (phage-encoded) gpL15 was replaced by the host-encoded gpL15-H protein properly due to its high level of expression prior to infection.

Immunological detection of gpL15-H incorporated into chimeric phage particles. Proteins within SDS-PAGE gels were transferred to 0.45 μm Invitrolon PVDF membranes (Invitrogen, Carlsbad, Calif.) using the Trans-Blot Semi-Dry Transfer Cell (Bio-Rad) according to the manufacturer's instructions. The presence of the hexahistidine tag was confirmed using the WesternBreeze Chromagenic Kit (Invitrogen, Carlsbad, Calif.). Typical results are shown in FIG. 3. During western analysis, mouse anti-hexahistidine IgG, antibodies (Roche) were used as the primary antibody, while alkaline phosphatase-conjugated anti-mouse IgG anti-bodies (Invitrogen, Carlsbad, Calif.) were used during chromogenic detection. As expected, gpL15-H was detected only in phages propagated on isolates of MG1363 (pJMS245::I15-H) (Lanes 3-4) and as a visible, but weak band in total proteins extracted from phage uninfected control cultures of MG1363 (pJMS245::I15-H) (Lanes 7-8). As seen in SDS-PAGE gels, the intensity of the gpL15-H band in Lanes 3-4 is greater than those in Lanes 7-8, indicating that the gpL15-H protein was concentrated through incorporation into the phage particles.

### Example 3

### Incorporation of the gpL15-H Antigen into Chimeric φc2 Particles Devoid of the gpL15-H Encoding Gene and Contiguous Plasmid DNA

Evaluation of plasmid transduction frequencies. Transduction studies were conducted as described by Birkeland and Holo (1993) in order to estimate the frequency with which the host-encoded plasmid, which encodes the antigenic recombinant fusion protein, is erroneously incorporated into phage particles (Table 2). MG1363 is naturally chloramphenicol sensitive, its frequency of spontaneous mutation to chloramphenicol resistance was found to be extremely low at a concentration of 5 μg/mL (<1.9×10⁻⁹). Sterile-filtered phages previously propagated on L. lactis MG1363 (pJMS245), MG1363 (pJMS245::I15) and MG1363 (pJMS245::I15-H) were incubated in the presence of MG1363 and the frequency in which MG1363 was converted to a chloramphenicol resistant phenotype was assayed. In all cases, no chloramphenicol resistant transductants were observed. These results suggest that the present system is an excellent platform for the production of chimerically labeled phage particles that are devoid of the cognate host-encoded nucleic acid coding for the antigen of interest.

TABLE 2
Frequencies of plasmid transduction^{a}.

| | | No. of chloramphenicol resistant | |
|---|---|---|---|
| | | transductants per: | |
| Plasmid | PFU/mL | mL | PFU |
| pJMS245 | 6.3 × 10⁸ | 0 | <1.6 × 10⁻⁹ |
| pJMS245::l15 | 7.0 × 10⁸ | 0 | <1.4 × 10⁻⁹ |
| pJMS245::l15-H | 5.5 × 10⁸ | 0 | <1.8 × 10⁻⁹ |

^{a}Each result in an average of two independent experiments.

### Example 4

### Construction of a Host Cell Suitable for the Conditional Production of Chimeric Particles

Construction of a pH-inducible gpL15-H expression system. PstI restriction endonuclease recognition sites were incorporated into the 5′ ends of primers JS14_F-pH and JS15_R-pH. A 118 bp fragment containing the pH-inducible promoter P170 (GenBank Accession Number AJ011913) was amplified from the vector pAMJ586 (Madsen et al., 1999) using JS14_F-pH and JS15_R-pH. The PCR fragment was restricted with PstI and ligated into the PstI site of pJMS124, a shuttle vector able to replicate in both E. coli and Lactococcus, that encodes erythromycin resistance as a selectable marker. The resulting plasmid DNA was purified and independently electroporated into electocompetent E. coli. Erythromycin resistant CFUs were screened for the presence of pJMS124::P170. The orientation of the P170 insert was confirmed by PCR using pJMS124-specific primers M13F or M13R in combination with primer JS14_F-pH.

Primers JS16_F-gpL15 and JS17_R-gpL15 were used to amplify a wild-type 115-containing PCR fragments from the genome of L. lactis φc2 (control). As described above, primers JS16_F-gpL15 and JS36_R-gpL15-H were again used to amplify a recombinant version of the 115 gene encoding gpL15-H. These DNA fragments were restricted with BamHI and independently ligated to BamHI restricted pJMS124. The resulting plasmid DNAs were purified and electroporated into electocompetent E. coli and erythromycin resistant CFUs were screened for the presence of either pJMS124::I15 or pJMS124::I15-H. Recombinant plasmids found to contain the inserts in the sense orientation were independently electroporated into L. lactis MG1363. Erythromycin resistant CFUs were screened for the presence of either pJMS124::P170::I15 or pJMS124::P170::I15-H. Using this system, transcription from the pH inducible promoter can be stimulated by reducing the pH of the culture medium to pH 5.5 for 20 with lactic acid for 20 minutes as described elsewhere (Madsen et al., 1999). Following a period of induced protein translation, the pH can then be elevated back to pH 7.0 and the equilibrated cultures infected with φc2 at a MOI of 0.1. Chimerically labeled phages can thus be produced if the lytic infection is allowed to proceed for at least one lytic cycle or until the culture is completely lysed.

JS14_F-pH (SEQ ID No. 6)
5′-AAAACTGCAGGAACTATGAATATCCACTCC-3′
JS15_R-pH (SEQ ID No. 7)
5′-AAAACTGCAGTAGACAACAAAATAGTAGAAG-3′
M13F (SEQ ID No. 8)
5′-GTAAAACGACGGCCAGT-3′
M13R (SEQ ID No. 9)
5′-AACAGCTATGACCATG-3′

### Example 5

### Competitive Exclusion of Escherichia coli strains expressing F18-type Fimbriae

To illustrate the principle of competitive exclusion the following experiment is contemplated using Escherichia coli strains expressing F18-type fimbrae as the model of a pathogenic organism.

Escherichia coli strains expressing F18-type fimbriae (ECF18), which are known to cause post-weaning diarrhea and edema disease in pigs (Ha, et al. 2003). More specifically, the F18 adhesin (FedF) (which is located at the tip of these fimbriae) is responsible for mediating specific attachments to the porcine gastrointestinal mucosa (Smeds et al., 2001). In this experiment a functional form of FedF will be surface displayed on chimeric particles according to the invention. Pigs will be fed these particles in variable amounts to observe if the particles competitively exclude ECF₁₈ bacteria.

In the experiment a vector that expresses a fusion protein comprising the FedF adhesin (from the F18+pig-pathogen Escherichia coli F107/86) and the gpL15 capsid protein from Lactococcus lactis phage c2 is constructed. The complete fedF gene could be fused to 5′ or 3′ of the complete I15 gene by SOEing PCR (Horton, R. M., 1995). The fusion allele(s) is then ligated to pJMS124::P170(3α) using T4 DNA ligase (Gibco-BRL Life Technologies, Inc.) according to the manufacturer's directions. The resulting plasmid (pJMS124::P170::I15::fedF) DNA is purified using the MiniElute Kit according to the manufacturer's specifications (Qiagen). This ligation will then be electroporated into electocompetent Escherichia coli (Bio-Rad Laboratories) according to the manufacturer's instructions using a Bio-Rad Gene Pulser (Bio-Rad Laboratories) and the method of Sambrook et al., (1989). Erythromycin resistant colonies will then be screened for the presence of pJMS124::P170::I15::fedF. These plasmids will then be electroporated into Lactococcus lactis MG1363 as described by Holo and Nes (1995). Chimeric phages will be produced according to the method described in Example 2 (above) except that pJMS124::P170::I15::fedF is used.

The resulting chimeric phages will be purified and fed to pigs (test) in defined amounts over a defined period of time. A second group of pigs (control) will not be fed the chimeric phages, but will instead be given an equivalent dose of sterile saline. Both groups of pigs (test and control) will then be inoculated with defined doses of Escherichia coli F107/86. Following inoculation, the symptoms of post-weaning diarrhea and edema disease will be monitored. The excretion of chimeric particles will be monitored over time by western hybridization and the shedding of E. coli F107/86 will be monitored by quantitative PCR.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references cited in this patent document are hereby incorporated herein in their entirety by reference.

### REFERENCES

Ausubel, F. M., et al. (eds.). “Current Protocols in Molecular Biology”. John Wiley and Sons. 1995.
Birkeland, N. and Holo, H. 1993. Transduction of a plasmid carrying the cohesive end region from Lactococcus lactis bacteriophage φLC3. Appl. Environ. Microbiol. 59: 1966-1968.
Blake, D. P., Hillman, K., Fenlon, D. R., Low, J. C. (2003) Transfer of antibiotic resistance between commensal and pathogenic members of the Enterobacteriaceae under ileal conditions. J. Appl. Microbiol. 95:428-36.
Charlier G, Bertschinger H U, Wild P, Vandekerckhove J, et al. 1993. The role of adhesive F107 fimbriae and of SLT-IIv toxin in the pathogenesis of edema disease in pigs. Zentralbi Bakteriol. 278(2-3):445-50.
Davis, S. S. (2001) Nasal vaccines. Adv. Drug Deliv. Rev. 51:21-24.
Elmer, G. W. (2001). Probiotics: “living drugs”. Am J Health Syst Pharm. 58(12):1101-9). FAO/WHO joint Expert Consultation on Evaluation of Health and Nutritional Properties of Probiotics in Food Including Powder Milk with Live Lactic Acid Bacteria, October 2001. http://www.mesanders.com/probio_report.pdf.
Foss, D. L. and Murtaugh M P. (2000) Mechanisms of vaccine adjuvanticity at mucosal surfaces. Anim. Health Res. Rev. 1:3-24.
Fuller, R. (1989) Probiotics in man and animals. J Appl Bacteriol 66: 365-78.
Gasson, M. J. 1983. Plasmid complements of Streptococcus lactis NCDO712 and other lactic streptococci after protoplast-induced curing. J. Bacteriol. 154:625-629.
Ha S K, Choi C, Chae C. (2003) Prevalence of a gene encoding adhesin involved in diffuse adherence among Escherichia coli isolates in pigs with postweaning diarrhea or edema disease. J Vet Diagn Invest. 15(4):378-81.
Holo H, Nes IF. 1995. Transformation of Lactococcus by electroporation. Methods Mol. Biol.; 47:195-9.
Holo, H., and I. F. Nes. 1989. High-frequency transformation, by electroporation, of Lactococcus lactis subsp. cremoris grown with glycine in osmotically stabilized media. Appi. Environ. Microbiol.
Horton, R. M. (1995) PCR-mediated recombination and mutagenesis: SOEing together tailor-made genes. Mol. Biotechnol. 3:93-99.
Huynh, T. V., R. A. Young, and R. W. Davis. 1985. Construction and screening cDNA libraries in λgt10 and λgt11. In DNA cloning, vol. D. M. Glover (ed.). Oxford: IRL Press Ltd., pp. 49-78.
Imberechts H, de Greve H, Hernalsteens J P, Schlicker C, Bouchet H, Pohl P, Johansen, E. (1999) Genetic engineering (b) Modification of bacteria. In Encyclopedia of Food Microbiology (Robinson, R., Baft, C. and Patel, P., eds.). Academic Press, London, pp. 917-921.
Lachman, L. et al (ed.) (1986) The Therory and Practice of Industrial Pharmacy. Third Edition. Lea & Fibiger, Philadelphia.
Laulund, S. (1994) Commercial aspects of formulation, production and marketing of probiotic products. In Human Health: The contribution of microorganisms. pp. 158-173. Gibson, S. A. W. (Ed.). Springer-Verlag, London.
Lubbers, M., Waterfield, N., Beresford, T., Le Page, R., and Jarvis, A. 1995. Sequencing and analysis of the prolate-headed lactococcal bacteriophage c2 genome and identification of the structural genes. Appl. Environ. Microbiol. 61:4348-4356.
Madsen S M, Arnau J, Vrang A, Givskov M, Israelsen H.1999. Molecular characterization of the pH-inducible and growth phase-dependent promoter P170 of Lactococcus lactis. Mol Microbiol. 32:75-87.
Mercer D. K. et al., (1999) Fate of free DNA and transformation of the oral bacterium Streptococcus gordonii DL1 by plasmid DNA in human saliva. Appl. Environ. Microbiol. 65:6-10.
Mogensen, G. et al. (2002) Inventory of Microorganisms with a documented history of use in food. Bulletin of the international Dairy Federation, No. 377 page 10-19
O'Sullivan, D. J., and T. R. Klaenhammer. 1993. Rapid mini-prep isolation of high-quality plasmid DNA from Lactococcus and Lactobacillus spp. Appl. Environ. Microbiol. 59:2730-2733.
Salminen S, et al. (1998). Demonstration of safety of probiotics—A review. Int J Food Microbiol, 44(1-2): 93-106.
Salminen, S. (2001), Scandinavian Journal of Nutrition/Näringsforskning Vol 45:8-12, 2001.
Sambrook, J., E. F. Fritsch, and T. Maniatis. 1982. Molecular cloning: a laboratory manual, 2^{nd} ed. Spring Harbor Laboratory, Cold Spring Harbor. N.Y.
Smeds A, Hemmann K, Jakava-Viljanen M, Pelkonen S, lmberechts H, Palva A. (2001) Characterization of the adhesin of Escherichia coli F18 fimbriae. Infect Immun. 69(12):7941-5.
Stuer-Lauridsen, B., Janzen, T., Schnabl, J., and Johansen, E. 2003. Identification of the host determinant of two prolate-headed phages infecting Lactococcus lactis. Virology 309:10-17.
Sturino J M, Klaenhammer T R. (2002) Expression of antisense RNA targeted against Streptococcus thermophilus bacteriophages. Appl Environ Microbiol. 68:588-96

1. A method of production of a composition comprising a chimeric phage-derived particle said method comprises
introducing into (eg. by transfection, infection and/or otherwise transformation) a safe host cell one or more genetic elements, which alone or in combination encodes the phage-derived particle.

2. A method of production of a composition comprising a chimeric phage-derived particle according to claim 1, wherein the safe host cell is selected from the group of bacteria consisting of bacteria the use of which have been evaluated by the United States Food and Drug Administration, Center for Veterinary Medicine to be generally recognized as safe (GRAS) and bacteria that, according to European Food and Fed Cultures Association and International Dairy Federation (EFFCA/IDF) are microorganisms with a documented history of use in food without adverse effects.

3. A method of production of a composition comprising chimeric phage-derived particles (such as chimeric phage particles, chimeric phage-like particles or chimeric phage ghost particles) that comprise at least two non-identical surface displayed proteins, said method comprising the steps of:
(i) obtaining at least two genetic elements wherein at least one of said genetic elements (the founder genetic element) comprises a substantial part of a phage genome and wherein the at least one other of said genetic elements (the trans-complementing genetic element) codes for the synthesis of at least one component (the additional component) that is directed to the surface of said particle during the assembly and/or release of the particle;
(ii) transfecting, infecting and/or otherwise transforming a suitable bacterial host cell with said two or more genetic elements, said host cell is a safe host cell (such as a cell selected from the group of bacteria consisting of bacteria the use of which have been evaluated by the United States Food and Drug Administration, Center for Veterinary Medicine to be generally recognized as safe (GRAS) and bacteria that, according to European Food and Fed Cultures Association and International Dairy Federation (EFFCA/IDF) are microorganisms with a documented history of use in food without adverse effects;
(iii) culturing said bacterial host cell under conditions that permit the expression of phage structural proteins encoded by said founder genetic element and the expression of said at least one additional component that is directed to the surface of said particle during the assembly and/or release of the particle;
(iv) subjecting said culture of bacterial host cells to conditions that results in formation of particles that comprise at least one additional component that is encoded by the trans-complementing genetic element but do not comprise the sequence encoding for, at least part of, said at least one additional component that is encoded by the trans-complementing genetic element; and
(v) obtaining a composition comprising chimeric phage-derived particles.

4. A method according to claim 3, wherein said genetic elements are introduced into the host cells by a method comprising the steps of:
(i) transfecting, infecting and/or otherwise transforming a suitable host cell with said at least one trans-complementing genetic element;
(ii) transfecting, infecting and/or otherwise transforming the host cell with said founder genetic element.

5. A method according to claim 1, wherein said transformation of a suitable bacterial host cell with said two or more genetic elements comprises cell fusion.

6. A method according to claim 1, wherein said chimeric phages-derived particles comprise several normal (such as two, three or more wild-type) phage components.

7. A method according to claim 1, wherein said founder genetic element replicates during the method.

8. A method according to claim 1, wherein said component is fusion protein and wherein said fusion protein is a translational fusion between a sequence that codes for a protein or peptide that directs the fusion protein to the surface of said chimeric phage-derived particle and an unrelated protein or peptide coding sequence.

9. A method according to claim 1, wherein said at least one other genetic element is selected from the group of genetic elements consisting of plasmids, transposons, prophages, prophage remnants, pseduophage, episomes and phagemids.

10. A method according to claim 1, wherein at least one of said at least two genetic elements is transferred to said bacterial host cell by phage infection.

11. A method according to claim 1, wherein said bacterial host cell is selected form the group of bacteria the use of which have been evaluated by the United States Food and Drug Administration, Center for Veterinary Medicine to be generally recognized as safe (GRAS) and are approved for use as additives for use in food, feed, or direct-fed microbial (DFM) products.

12. A method according to claim 11, wherein said bacterial host cell is regarded GRAS with respect to their use in dairy food products

13. A method according to claim 1, wherein said bacterial host cell is selected form the group of bacteria consisting of bacteria, that according to European Food and Fed Cultures Association and International Dairy Federation (EFFCA/IDF) are microorganisms with a documented history of use in food without adverse effects.

14. A method according to claim 1, wherein the host cell is selected from the group of lactic acid bacteria.

15. A method according to claim 1, wherein the host cell is selected from the group of non-pathogenic bacteria genera consisting of non-pathogenic Arthrobacter spp., Bifidobacterium spp., Brevibacterium spp., Corynebacterium, Enterobacter spp., Enterococcus spp., Hafnia spp., Kocuria spp., Lactobacillus spp., Lactococcus spp., Leuconostoc spp., Micrococcus spp., Oenococcus spp., Pediococcus spp., Propionibacterium spp., Rhodosporidium spp., Staphylococcus spp. and Streptococcus spp.

16. A method according to claim 1, wherein the host cell is selected from the group of non-pathogenic bacteria consisting of Arthrobacter globiformis, Bifidobacterium adolescentis, Bifidobacterium animalis (previously Bifidobacterium bifidum), Bifidobacterium breve, Bifidobacterium infantis, Bifidobacterium lactis, Bifidobacterium longum, Bifidobacterium pseudolongum, Bifidobacterium thermophilus, Brevibacterium casei, Brevibacterium linens, Corynebacterium flavescens, Enterococcus aerogenes, Enterococcus faecium, Hafnia alvei, Kocuria varians, Lactobaccillus delbrueckii subsp. lactis, Lactobacillus acidophilus, Lactobacillus alimentarius, Lactobacillus alimentarius brevis var. lindneri, Lactobacillus bavaricus, Lactobacillus brevis, Lactobacillus brevis var. lindneri, Lactobacillus bulgaricus, Lactobacillus carnis, Lactobacillus casei subsp. casei, Lactobacillus casei var. rhamnosus, Lactobacillus cremoris, Lactobacillus curvatus, Lactobacillus delbrueckii, Lactobacillus delbrueckii subsp. bulgaricus, Lactobacillus delbrueckii subsp. lactis, Lactobacillus farciminis, Lactobacillus helveticus, Lactobacillus jensenii, Lactobacillus lactis, Lactobacillus lactis subsp. lactis, Lactobacillus lactis subsp. lactis biov. diacetyllactis, Lactobacillus leichmanii, Lactobacillus paracasei (previously Lactobacillus casei), Lactobacillus paracasei paracasei, Lactobacillus paracasei subsp. paracasei, Lactobacillus pentosus, Lactobacillus plantarum, Lactobacillus rhamnosus, Lactobacillus sake (earlier L. alimentarius), Lactobacillus sanfrancisco, Lactobacillus xylosus, Lactococ lactis sub. lactis biovar. diacetylactis, Lactococcus (formerly Streptococcus) lactis subsp. cremoris, Lactococcus acidophilus, Lactococcus lactis, Lactococcus lactis spp. diacetilactis (previously Streptococcus diacetilactis), Lactococcus lactis subsp. cremoris, Lactococcus lactis subsp. lactis, Lactococcus lactis subsp. lactis diacetylactis, Leuconostoc carnosum, Leuconostoc citrivorum, Leuconostoc dextranicum, Leuconostoc mesenteroides subsp. cremoris, Leuconostoc pseudomesenteroides, Micrococcus varians, Oenococcus oeni (previously Leuconostoc oenos), Pediococcus acidilactici, Pediococcus pentosaceus, Propionibacterium acidipropionici, Propionibacterium arabinosum, Propionibacterium freudenreichii ssp. Spermanii, Propionibacterium freudenreichii, Propionibacterium shermanii, Rhodosporidium infirmominiatum, Staphylococcus carnosus, Staphylococcus xylosus, Streptococcos salivarius subsp. thermophilus, Streptococcus cremoris, Streptococcus diacetylactis, Streptococcus durans, Streptococcus faecium, Streptococcus lactis, Streptococcus thermophilus (previously Streptococcus salivarius subsp. thermophilus), Bacillus coagulans, Bacillus lentus, Bacillus licheniformis, Bacillus pumilus, and Bacillus subtillis.

17. A method according to claim 1, wherein the host cell prior to the addition of said two or more genetic elements can be regarded as a microorganism or food-grade GMO as defined by Johansen (1999).

18. A method according to claim 1, wherein said chimeric phages-derived particles that do not comprise the sequence encoding for at least part of said fusion protein are released from said bacterial host cells by the action of one or more phage- and/or host-genome-encoded component(s).

19. A method according to claim 18, wherein said phage-genome encoded component or components comprise holin and/or endolysin and/or lysozyme.

20. A method according to claim 1, wherein said chimeric particles are released from said bacterial host cell by non-phage-induced lysis, including the physical disruption processes (e.g. sonication, French press, beads, grinding, etc.) and/or the addition of chemicals (e.g. phage lysins, lysozyme, etc.).

21. A method for obtaining a chimeric phage-derived particle (such as a chimeric phage, chimeric phage-like or chimeric phage ghost particle) that comprise at least two different surface displayed proteins, said method comprising the steps of:
(i) obtaining a composition from where said chimeric phage-derived particle may be isolated according to claim 1,
(ii) isolate said chimeric phages-derived particle from said composition.

22. A method according to claim 1 for the production of a chimeric phage-derived particle by traditional batch fermentation.

23. A method according to claim 1 for the production of a chimeric phage-derived particle by continuous fermentation, including immobilized cell technology.

24. A chimeric phage-derived particle which is obtainable by a method of claim 1.

25. A chimeric phage-derived particle (such as a chimeric phage, chimeric phage-like or chimeric phage ghost particle) that:
in addition to at least one normal phage component displays at least one additional component, said at least one normal phage component being coded by a genetic element that comprises a substantial part of a phage genome the founder genetic element and said at least one additional component being coded by a different genetic element the trans-complementing genetic element;
is further characterized in that it does not comprise the sequence encoding for at least part of said at least one additional component; and
is produced by use of a safe host cell, such as a cell which are selected form the group of bacteria consisting of bacteria the use of which have been evaluated by the United States Food and Drug Administration, Center for Veterinary Medicine to be generally recognized as safe (GRAS) and bacteria, that according to European Food and Fed Cultures Association and International Dairy Federation (EFFCA/IDF) are microorganisms with a documented history of use in food without adverse effects.

26. A particle according to claim 24, wherein said particle in addition to several normal phage components display at least one additional component.

27. A particle according to claim 24, wherein said particle do not comprise the any sequence encoding for said at least one additional component.

28. A particle according to claim 24, wherein said at least one additional component being coded by a different genetic element is a fusion protein being a fusion between a peptide sequence that direct the fusion protein to the surface of said particle and an unrelated peptide sequence.

29. A according to claim 28, wherein said fusion protein comprise a peptide sequence comprising a functional part of a phage (capsid) protein.

30. A particle according to claim 29, wherein said phage (capsid) protein is a component of a phage head, a phage prohead, a phage collar, a phage whisker, a phage tail, a phage base plate, and/or a phage tail fiber.

31. A particle according to claim 30, wherein said phage protein is selected from the group of phage proteins consisting of gpL1, gpL2, gpL3, gpL4, gpL5, gpL6, gpL7, gpL8, gpL9, gpL10, gpll1, gpL12, gpL13, gpL14, gpL15, gpL16, and gpL17 derived from phages similar to the lactococcal type phage c6A, including phage c2.

32. A particle according to claim 24, wherein said particle is infective.

33. A particle according to claim 24, wherein said particle is infective and exhibits a host specificity that is determined by said genetic element that comprises a substantial part of a phage genome (the founder genetic element).

34. A particle according to claim 24, wherein said particle exhibits a host specificity that is determined by said at least one different genetic element (the trans-complementing genetic element).

35. A particle according to claim 24, wherein said host specificity is retained and identical to the naturally occurring phage isolate from where said substantial part of a phage genome was derived (i.e. the “founder particle”).

36. A particle according to claim 24, wherein said host specificity is altered relative to the naturally occurring phage isolate from where said substantial part of a phage genome was derived.

37. A particle according to claim 24, wherein said particle exhibits an increased or a reduced capacity to infect bacteria relative to the naturally occurring phage isolate from where said substantial part of a phage genome was derived (the founder genetic element).

38. A particle according to claim claim 22, wherein said particle is not infective.

39. A particle according to claim 1, which particle does not contain any genetic material.

40. A particle according to claim 1, wherein said fusion protein is able to associate with virus-encoded components.

41. A particle according to claim 40, wherein said fusion protein is able to associate with virus-encoded proteins comprised in the naturally occurring phage isolate from where said substantial part of a phage genome was derived (the founder particle).

42. A particle according to claim 41, wherein said fusion protein is able to associate with said virus-encoded one or more proteins of said naturally occurring phage isolate prior to lysis of the bacterial host cell.

43. A particle according to claim 41, wherein said fusion protein is able to associate with said virus-encoded one or more proteins of said naturally occurring phage isolate after said chimeric particles are released form the host cell.

44. A according to claim 1, wherein in addition to said at least one normal phage component the particle comprise at least two additional components that are not encoded by said genetic element that comprises a substantial part of a phage genome.

45. A particle according to claim 28, wherein said unrelated peptide sequence of said fusion protein is derived from the genome of plants, humans, animals, fungi, bacteria, or viruses.

46. A particle according to claim 28, wherein said unrelated peptide sequence of said fusion protein is derived from a pathogen of plants, humans, animals, fungi, or bacteria.

47. A according to claim 28, wherein said unrelated peptide sequence of said fusion protein is derived from a microorganism whose interaction with plants, humans, animals, fungi, or bacteria, may be considered non-beneficial but not pathogenic.

48. A particle according to claim 28, wherein said unrelated peptide sequence of said fusion protein encodes a virulence factor comprised of a specific sequence of amino acids or a portion thereof

49. A particle according to claim 28, wherein said unrelated peptide sequence of said fusion protein encodes a protein or peptide that facilitates and/or enables the binding of the particle to receptors found on a solid surface, a biofilm, human or animal cells, or other microbes.

50. A particle according to claim 28, wherein said unrelated peptide sequence of said fusion protein comprise a protein or peptide sequence (e.g. poly histidine) that facilitates and/or enables the conditional binding of the particle to a matrix for the purification of the said particle.

51. A particle according to claim 28, wherein said unrelated peptide sequence of said fusion protein encodes an antigen and/or allergen able to elicit an immune response in humans and/or animals

52. A particle according to claim 28, wherein said unrelated peptide sequence of said fusion protein is a peptide that enables the specific binding of at least one molecule.

53. A particle according to claim 51, wherein the at least one molecule that binds to said fusion protein is a protein, lipoprotein, glycoprotein, carbohydrate, lipid, or similar molecule of biological origin.

54. A particle according to claim 52, wherein said at least one molecule that binds to said fusion protein functions as an extra cellular receptor.

55. The use of a chimeric phage-derived particle according to claim 1 to produce a vaccine.

56. The use of a particle according to claim 1 to produce an immunostimulatory adjuvant.

57. The use of a particle according to claim 1 to produce a composition that comprise specific antigens to the immune system of an organism selected from the group consisting of mammals, fish and birds

58. The use of a particle according to claim 1 to produce a composition that competitively exclude pathogens or non-desirable microorganisms

59. The use of a particle according to claim 1 to produce a composition that competitively exclude pathogens or non-desirable microorganisms associated with mucosal surfaces, including the conjunctiva, the gastrointestinal tract, the respiratory tract, and the urogenital tract of humans and/or animals.

60. The use of a particle according to claim 1 to produce a composition that competitively excludes pathogens or non-desirable microorganisms, associated with plants and/or weeds relevant to human agriculture.

61. The use of a particle according to claim 1 to produce a probiotic and/or prebiotic composition.

62. The use of a particle according to claim 1 to produce a direct-fed microbial composition.

63. A composition comprising a chimeric phage-derived particle (such as a chimeric phage, chimeric phage-like or chimeric phage ghost particle) according to claim 1 useful for phage therapy.

64. A composition comprising a particle according to claim 1 that is useful as a biocontrol agent to control the number of specific pathogenic and/or non-desirable microorganisms.

65. A composition comprising a particle according to claim 1 that is useful to neutralize, kill and/or impede, a pathogen or non-desirable microorganism by means other than those associated with conventional phage therapy or phage biocontrol through the delivery of one or more cytotoxic agent(s).

66. A composition comprising a particle according to claim 1 that is useful to neutralize, kill and/or impede, a pathogen or non-desirable microorganism by means other than those associated with conventional phage therapy or phage biocontrol by precluding the pathogen or non-desirable microorganism from associations that normally allow for the deleterious characteristics in vivo.

67. A composition comprising a particle according to claim 1 that is useful for the treatment of allergies.

68. A composition comprising a particle according to claim 1 that is useful for the binding and/or neutralization of biological toxins.

69. A composition comprising a particle according to claim 1 that displays an additional tag that facilitates their binding and/or downstream purification.

70. Chimeric phage-derived particle (such as a chimeric phage, phage-like or phage ghost particle) according to claim 1 that surface displays one or more unrelated peptide sequences that acts as a generic adapter for the non-covalent binding of heterologous bioactive molecule(s) following purification of said particle.

71. Chimeric phage-derived particle according to claim 1 that surface displays one or more unrelated peptide sequences that acts as a generic adapter that facilitates the covalent linkage of one or more heterologous bioactive molecules by chemical or enzymatic treatment following purification of said particle.


SEQUENCE LISTING (9 sequences)

SEQ ID NO: 1 - DNA, 1388 bp, L. lactis fc2 phage
ttttgtctctcctagttgtacctcatgtcgtaaggcaagagcatggcttgaaaaacataatattccatttaaggaaagaaacattttttctgagccattaactaaagaagaattattaaagatcctctagagtcgacctgcagccaagctttcgggggatccagatctcacaatagaaagggtatataaatgatttcatggttaaatttcgaggagttattaattcataaccctattgagttgataaaccctagtaaagatacaataagagtagcaatgagccaaaagcagtatatcgagtttttcagcaataaatacacttataacggtctgtattatgacgaagaaatggacttctgtttattttattatgcagacccattacaaagctacaaagagggcgatgtgtacgctcaaggatatattgatgtagaaatgaaaatataccgcgtaaaatggttgtgtaacgtttctattagtcgtcctagtggtttattgcaaactactgacggaacccaaggagtgccacaagagggcggaaaatacactcacacagcatggtcttacagtgcagacggtacagacagattctcaactgtttatcctaatttgaatttattgaatgggactaaagattttaatggggattggataaatggaggtgtttggggaaacgatggaaaatataaaggcttaactgttaaaagttatcaaaaagcatgggacggaatgttcaaaaaatatattgtcccacaagacggtttatatacatggtctagttttgtcaagagtgaatcagacacctctgacatttttagagtattgttcataaataataaggaatttcctattgttgggcttggtcataaatttgattggcttcgtgattccgtaacagtacctctaaaaaaaggcgatgaagtcatatttaactatggtaatttaaaaaataatggaggtaaattaagtgttgctggttataaactagaatcaggttcaattgccactccttggatgccctcagctagcgaagtcacaacttctgacggtcctagctacatcggtcaatatacagattacacgctagaggacagtacaaaccctagttcttacacttggagagaaatacgagaggacaaatggaacgttacaaaaataggtatgcttgtaagtccacaagataaacaaattacaatggttcaagcaggggcattaatgaggtgtggaattaataacgacattacaggttggacagacggaacaacacaattaaattacagcggtcaagattttataattgacggttatggaatgagagggctacacaatggacatcatcatcatcatcattagcccgggggatccgtcgacctgcagccaagctttcg

SEQ ID NO: 2 - protein, 387 aa, L. lactis fc2 phage
MISWLNFEELLIHNPIELINPSKDTIRVAMSQKQYIEFFSNKYTYNGLYYDEEMDFCLFYYADPLQSYKEGDVYAQGYIDVEMKIYRVKWLCNVSISRPSGLLQTTDGTQGVPQEGGKYTHTAWSYSADGTDRFSTVYPNLNLLNGTKDFNGDWINGGVWGNDGKYKGLTVKSYQKAWDGMFKKYIVPQDGLYTWSSFVKSESDTSDIFRVLFINNKEFPIVGLGHKFDWLRDSVTVPLKKGDEVIFNYGNLKNNGGKLSVAGYKLESGSIATPWMPSASEVTTSDGPSYIGQYTDYTLEDSTNPSSYTWREIREDKWNVTKIGMLVSPQDKQITMVQAGALMRCGINNDITGWTDGTTQLNYSGQDFIIDGYGMRGLHNGHHHHHH

SEQ ID NO: 3 - DNA, 39 bp, artificial
  PCR primer JS16_F-gpL15
cgcggatccagatctcacaatagaaagggtatataaatg

SEQ ID NO: 4 - DNA, 35 bp, artificial
  PCR primer JS17_R-gpL15
cgcggatccagatctctatccattgtgtagccctc

SEQ ID NO: 5 - DNA, 60 bp, Artificial
  PCR primer JS36_R-gpL15-H
cgcggatcccccgggctaatgatgatgatgatgatgtccattgtgtagccctctcattcc

SEQ ID NO: 6 - DNA, 30 bp, Artificial
  PCR primer JS14_F-pH
aaaactgcaggaactatgaatatccactcc

SEQ ID NO: 7 - DNA, 31 bp, artificial
  PCR primer JS15_R-pH
aaaactgcagtagacaacaaaatagtagaag

SEQ ID NO: 8 - DNA, 17 bp, artificial
  PCR Primer M13F
gtaaaacgacggccagt

SEQ ID NO: 9 - DNA, 16 bp, Artificial
  PCR primer M13R
aacagctatgaccatg